(19) United States
(12) Patent Application Publication
(10) Pub. No.: US 2003/0129618 A1
(43) Pub. Date: Jul. 10, 2003

(54) Sensitive and rapid detection of pathogenic organisms and toxins using fluorescent polymeric lipids

(75) Inventors: Mario Manuel Moronne, Berkeley, CA (US); Deborah H. Charych, Albany, CA (US); Jon O. Nagy, Bozeman, MT (US)

Correspondence Address: MEDLEN & CARROLL, LLP, Suite 350, 101 Howard Street, San Francisco, CA, 94105, US

(73) Assignee: Regents of the University of California, Oakland, CA (US)

(21) Appl. No.: 10/215,736

(22) Filed: Aug. 9, 2002

Related U.S. Application Data
(60) Provisional application No. 60/311,779, filed on Aug. 10, 2001.

(51) Int. Cl. C12Q 1/70; C12Q 1/68; G01N 33/53; G01N 33/543

(52) U.S. Cl. 435/6; 435/7.1; 436/518; 435/5

(57) ABSTRACT

The present invention relates to methods and compositions for the detection of analytes using the fluorescence that occurs in polymeric material in response to selective binding of analytes to the polymeric materials. In particular, the present invention allows for the fluorescent detection of membrane modifying reactions and analytes responsible for such modifications and for the screening of reaction inhibitors.

This application claims priority to U.S. Provisional Application No. 60/311,779, filed Aug. 10, 2001.

This invention was made in part during work partially supported by the U.S. government Department of Energy grant No. DE-AC03-76SF00098. The government has certain rights in the invention.

### FIELD OF THE INVENTION

The present invention relates to methods and compositions for the detection of analytes using the fluorescence that occurs in polymeric material in response to selective binding of analytes to the polymeric materials. In particular, the present invention allows for the fluorescent detection of membrane modifying reactions and analytes responsible for such modifications and for the screening of reaction inhibitors.

### BACKGROUND OF THE INVENTION

A major goal of analyte detection research is to develop fast, reliable, and sensitive detectors. Unfortunately, the technologies developed to date have only met some of these goals, and no single device has sufficiently attained a majority of them.

Classical detection methods such as liquid chromatography (LC), gas chromatography (GC), and supercritical fluid chromatography (SFC), in combination with mass spectrometry, are widely used to provide identification of analytes and quantitative data. However, these techniques are time consuming, extremely expensive, require sample preconcentration, and are not sensitive enough for many applications.

The art remains in need of analyte detectors that provide ease of use and sensitivity that is lacking from available technologies. In particular, art remains in need of analyte detectors that allow for the detection of multiple analyte types, and for the detection of even trace amounts of analytes in a sample.

### SUMMARY OF THE INVENTION

The present invention relates to methods and compositions for the detection of analytes using the fluorescence that occurs in polymeric material in response to selective binding of analytes to the polymeric materials. In particular, the present invention allows for the fluorescent detection of membrane modifying reactions and analytes responsible for such modifications and for the screening of reaction inhibitors.

In particularly preferred embodiments, the polymeric materials comprise biopolymeric materials by the addition to or modification of the polymers with one or more biological molecules or groups (e.g., amino acid(s), nucleic acid(s), sugars, lipids, organometallic centers, etc.). In some embodiments, the monomers of the present invention are self-assembling. In other embodiments, the monomers are assembled using compression techniques (e.g., Langmuir trough). In certain of these embodiments, the polymeric (in some cases biopolymeric) films are over-compressed to form multi-layer arrangements (e.g., bilayers, trilayers, etc.). Similarly, in other embodiments, the polymeric materials of the present invention are assembled into mon- and/or multilayer thin films.

In some embodiments, the self-assembling monomers of the polymeric material of the presently claimed invention comprise diacetylene monomers. In certain embodiments, the diacetylene monomers are selected from the group consisting of 5,7-docosadiynoic acid, 5,7-pentacoadiynoic acid, 10,12-pentacosadiynoic acid, and combinations thereof, although all diacetylene monomers are contemplated by the presently claimed invention. In other embodiments, the self-assembling monomers are selected from the group consisting of acetylenes, alkenes, thiophenes, polythiophenes, imides, acrylamides, methacrylates, vinylether, malic anhydride, urethanes, allylamines, siloxanes anilines, pyrroles, vinylpyridinium, and combinations thereof. In certain embodiments, the self-assembling monomers contain head groups selected from the group consisting of carboxylic acid, hydroxyl groups, amine groups, amino acid derivatives, and hydrophobic groups, although other head groups are also contemplated by the presently claimed invention.

In other preferred embodiments, the polymeric materials comprise water-soluble glycopolythiophenes (e.g., containing sialic acid or mannose ligands) such materials have been synthesized by oxidative co-polymerization of methyl thiopheneacetate and thiophene-carbohydrate monomers. In some embodiments of the present invention, a carbohydrate ligand is placed next to the phenyl group of the glycopolythiophene assemblies to prevent neuraminidase cleavage of the O-linked glycosides of sialic acid and to provide tighter binding to Escherichia coli. Certain embodiments of the present invention are modified in this manner without losing binding properties.

The present invention provides biopolymeric materials comprising a plurality of polymerized monomers and one or more ligands, wherein the biopolymeric materials fluoresce in the presence of analyte. In some embodiments, the ligands are selected from the group consisting of peptides, proteins, antibodies, receptors, transmembrane channels, and combinations thereof, although the present invention specifically contemplates all protein ligands (i.e., with protein being defined in its broadest sense). In other embodiments, the ligands are non-proteins (e.g., lectins, carbohydrates, glycolipids, phospholipids, and the like). However, the present invention is not limited to any particular ligand-analyte binding partners.

In some preferred embodiments, protein ligands comprise antibodies or portions of antibodies, proteins, or polypeptides, and the like. In other preferred embodiments that employ non-protein ligands, a number of non-protein molecules are contemplated (e.g., altered naturally occurring nucleic acids, oligonucleotides, drugs, chromophores, antigens, chelating compounds, molecular recognition complexes, ionic groups, polymerizable groups, linker groups, electron donors, electron acceptor groups, hydrophobic groups, hydrophilic groups, receptor binding groups, polysacchrides (e.g., trisaccharides, tetrasaccharides, etc.) ganglioside G_{M1}, ganglioside G_{T1b}, sialic acid, and organic molecules capable of specifically binding to a receptor (e.g., cyclosporin, benzadiazapam, or serotonin uptake transporters, ACE), metal-complexes, and inorganic materials such as transition and lanthanide series metals. and combinations thereof).

In particularly preferred embodiments, the ligand is specifically varied according to the analyte to be detected.

In some embodiments, the inventive biopolymeric materials comprise three portions: a polymer, a spacer, and a ligand. Because of the adaptability of these assemblies, modifications may be made which give it great variety in application and design. The present invention is not limited to the following variations in some embodiments. Some preferred embodiments of the present invention employ variation in the polymer backbone, thereby producing different shapes of conjugation. This is accomplished through the addition of aromatic and or non-aromatic units (e.g., thiazole, pyrrole, selenophene phenyl unit, phenylene vinylene unit and diacetylene) as co-monomers without losing conjugation. Alternatively, the polymer backbone is altered by heterocyclic atoms used in place of carbon (e.g., N, O, and Se). Other embodiments of the present invention vary the length or composition of the spacer element. For example, almost any length spacer (e.g., one or more carbon atoms) having hydrophilic/lipophilic properties is permitted. The alteration of spacer length and composition is directed by observing the fluorescent responses obtained such that a desired response is reached. It is contemplated that the ability to vary spacer length and composition allows the polymer assemblies greater access to high molecular weight molecules (e.g., viruses, bacteria, and parasites). In some embodiments, neither the ligand, spacer, or polymer assembly comprises a lipid.

In alternative embodiments of the present invention, the portions of the ligand or monomer assemblies are manipulated to alter their shape and electronic conformation of the composition.

The biopolymeric materials of the presently claimed invention provide a means to fluorescently detect analytes. It is not intended that the present invention be limited to detecting any particular analyte. Indeed, in some embodiments, the analyte is selected from the group consisting of pathogens, drugs, receptor ligands, antigens, ions, hormones, blood components, disease indicators, cell components, antibodies, lectins, enzymes, organic solvents, volatile organic compounds, pollutants, and genetic material. In other embodiments, the analyte is a pathogen selected from the group consisting of viruses, bacteria, parasites, and fungi. In some embodiments, the pathogen is a virus selected from the group consisting of influenza, rubella, varicella-zoster, hepatitis A, hepatitis B, herpes simplex, polio, small pox, human immunodeficiency virus, vaccinia, rabies, Epstein Barr, reoviruses, and rhinoviruses. In other embodiments, the pathogen is a bacterium selected from the group consisting of E. coli, Mycobacterium tuberculosis, Salmonella, Chlamydia and Streptococcus. In yet other embodiments, the pathogen is a parasite selected from the group consisting of Plasmodium, Trypanosoma, Toxoplasma gondii, and Onchocerca or portions of these parasites.

Still further embodiments of the present invention employ one or more accessory molecules (i.e., dopants) that alter ligand-receptor binding or that alter the detectable response of the biopolymeric material. As used herein “alter ligand binding” encompasses both attenuating or enhancing ligand binding (the fluorescent response of the polymer assemblies upon the binding of an analyte may, or may not, be effected by the particular dopant selected).

In certain embodiments of the presently claimed invention, the biopolymeric materials further comprise one or more dopant materials. In some embodiments, the dopant material is selected from the group consisting of surfactants, polysorbate, octoxynol, sodium dodecyl sulfate, polyethylene glycol, zwitterionic detergents, decylglucoside, deoxycholate, diacetylene derivatives, phosphatidylserine, phosphatidylinositol, phosphatidylethanolamine, phosphatidylcholine, phosphatidylglycerol, phosphatidic acid, phosphatidylmethanol, cardiolipin, ceramide, cholesterol, steroids, cerebroside, lysophosphatidylcholine, D-erythroshingosine, sphingomyelin, dodecyl phosphocholine, and N-biotinyl phosphatidylethanolamine, although any other material that can be associated with the biopolymeric material of the presently claimed invention is contemplated. In specific embodiments, the dopant material is a diacetylene derivative selected from the group consisting of sialic acid-derived diacetylene, lactose-derived diacetylene, and amino acid-derived diacetylene, although all diacetylene derivatives are contemplated by the presently claimed invention.

The present invention contemplates a number of means, both covalent and noncovalent, for associating dopant molecules with the biopolymeric materials. For example, in some embodiments, dopant molecules are attached directly to the polymer assemblies, alternatively, these molecules are attached to the polymer assemblies via a linker. In still other embodiments, these molecules can be attached to the ligand molecules. In any event, the dopant molecules are positioned relative to the polymer assemblies such that a desired effect is exerted on ligand binding or fluorescent response. Other embodiments of the present invention employ alternative physical schemes for altering ligand binding and fluorescent response (e.g., heat, pressure, irradiation, and the like).

In particularly preferred embodiments of the present invention, the biopolymeric materials are designed to produce freely soluble polymers in aqueous solution and to increase inter-chain interactions of the compositions (e.g., polydiacetylenes, glycopolythiophenes, polythiophenes, and thiophenes). However, the present invention is not intended to be limited to any particular configuration. Various configurations are envisioned. In one embodiment, the polymer assemblies comprise linear glycopolymers. In other embodiments, the polymer assemblies are configured as films, or vesicles. Particularly preferred embodiments of the present invention comprise liposome based assay materials. Still further embodiments contemplate compositions and methods employing immobilized polymers on various solid substrates (e.g., including microtiter plates, tubes, beads, and wells).

In certain embodiments, the solid substrate is selected from the group consisting of polystyrene, polyethylene, teflon, mica, sephadex, sepharose, polyacrynitriles, filters, glass, gold, silicon chips, and silica. In other embodiments, the support comprises porous silica glass, wherein the biopolymeric materials are immobilized within the porous silica glass (e.g., sol-gel glass), although the presently claimed invention contemplates a variety of other supports. The presently claimed invention provides a device comprising one or more of the biopolymeric materials described above, wherein the biopolymeric materials are immobilized to the device.

The present invention is not intended to be limited to any particular method of polymer assembly or synthesis. Indeed, a number of polymer assembly synthesis techniques are contemplated.

It is not intended that the present invention be limited to polydiacetylene monomers. A variety of polymerizable monomers are contemplated. For example, in one embodiment, polymerized thiophene monomers are used. In another embodiment, polymerized polythiophene monomers are used.

In some particularly preferred embodiments of the present invention, the compositions further comprise one or more spacer molecules. Suitable spacer molecules can be hydrophilic or hydrophobic. In some of these embodiments, the spacer molecule comprise from 1-1,000 carbon atoms, preferably from 100-500 carbon atoms, more preferably from 20-50 carbon atoms, and most preferably from 5-10 carbon atoms. Additionally, in some embodiments, one or more covalent bonds attach one or more ligands to the biopolymeric materials (e.g., amine bonds, sulfide bonds, thiol bonds, aldehyde bonds, glycosidic bonds, and peptide bonds). Some also comprise one or more spacer molecules.

In preferred embodiments of the present invention, a fluorescent response is obtained from conformational changes in the biopolymeric assemblies. While an understanding of the mechanism is not important to practice of the present invention, the fluorescent response is believed to result from stress induced in the polymer through the binding of an attached ligand and its analyte.

The present invention also provides methods of detecting the presence of an analyte in a sample, comprising: a) providing: i) biopolymeric materials comprising a plurality of monomers and one or more ligands wherein said biopolymeric materials fluoresces in the presence of an analyte; and ii) a sample suspected of containing an analyte; b) contacting said biopolymeric materials with said sample; and c) detecting fluorescence in said biopolymeric materials. Indeed, the present invention contemplates various methods and kit embodiments for detecting the presence of analytes using the novel fluorescent materials disclosed herein. For example, the compositions and methods of the present invention are readily suited for assays employed to discover various reaction inhibitors. Moreover, the compositions and methods of the present invention are fully scaleable for uses requiring high throughput screening techniques such as drug development, analytical chemistry, genomics, and proteomics.

The present inventive assemblies can also be applied to the manufacture of environmental biosensors for the detection of air and water contaminants and contaminants in food and beverages.

Preferred embodiments of the fluorescent detection methods of the present invention are useful for the ultra-rapid and ultra-sensitive detection of single analyte molecules (e.g., single virus particles, single proteins molecules, and the like). The ultra-rapid advantages of the present invention make preferred embodiments suitable for real time analyses in a variety of industrial, military, medical, and domestic applications.

### DESCRIPTION OF THE FIGURES

FIGS. 1A and 1B show 250 by 200 μm domains illustrating the detection of influenza virus (FIG. 1B) versus a control (FIG. 1A). FIG. 1C, shown at higher resolution than FIGS. 1A and 1B, shows distinct fluorescent fingerlings (fluorescent reporter polymer units). Each unit, in effect represents one detector element. A 100 μm×100 μm domain will contain from 16,000 to 138,000 of the fluorescent reporter polymer units. Each activated domain produces greater than 20000 cps. In a preferred embodiment a scanning device including optical detectors reads about 100 domains in an estimated 3 sec. In particularly preferred embodiments, comprising fiber optic design elements the assay time is about 30 msec. FIGS. 1A-1C were taken using total internal reflection microscopy (TIR).

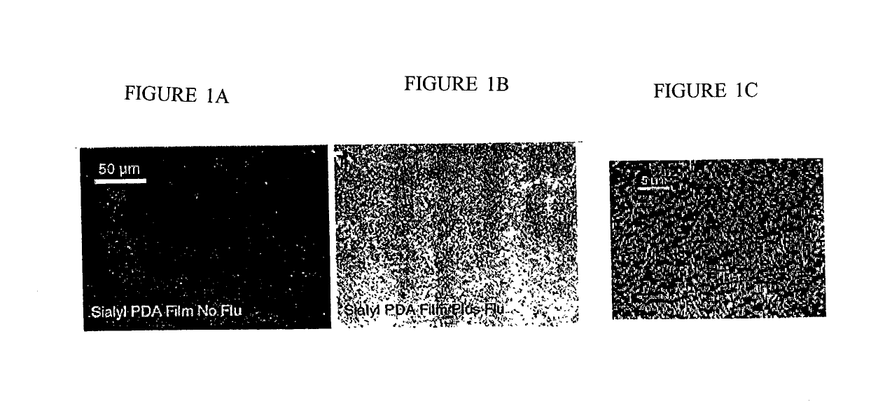

FIG. 2 shows one embodimnet of the present invention, wherein each bright spot in the figure was calibrated against the fluorescent dye rhodamine and correlated with the quantum yield sialyl-PDA vesicles. Analysis of the intensities indicate that each spot corresponds to from one to three vesicles of PDA. This shows that single virus particles can be detected using preferred embodiments of the compositions disclosed herein.

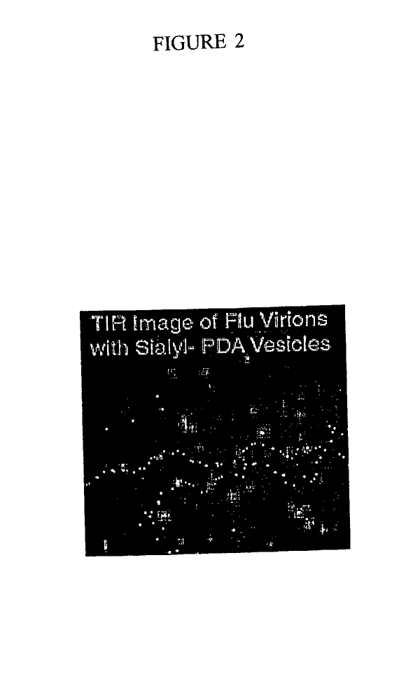

FIG. 3 compares the fluorescent response of a UV polymerized trimolecular polydiacetylene film containing 30% of sialyl polydiacetylene that strongly binds influenza virus. In the top two panels labeled controls, the polymer film is exposed to the viral support media but contains no virus particles. Very little fluorescence is seen. The bottom four frames labeled “plus flu” shows dramatic fluorescence generation when the influenza virus is included in the media. Each image frame is about 400 um across. The total fluorescence signal induced by virus binding corresponds to >than 2 billion counts. This corresponds to about 0.002% error in the signal. This value is thousands of times better than typical of biological assays used clinically and was acquired in under a minutes time compared to the hours or days needed for many clinical assays.

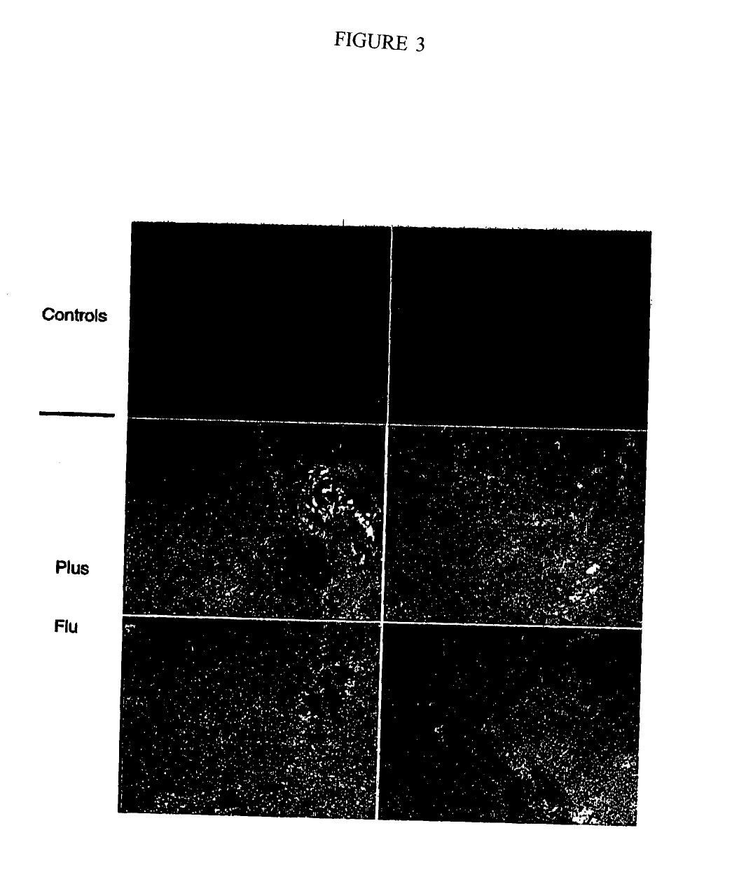

FIG. 4 illustrates the induction of polydiacetylene fluorescence in 50 to 100 nm size polymer vesicles when treated with influenza virus. The images were acquired using total internal reflection microscopy (TIR) and a very sensitive camera capable of detecting single fluorescent molecules. With appropriate calibration these images demonstrate that single virus particles can be detected by the fluorescence change induced in the polymer vesicles. In the left panel, particles are seen to fluoresce which correspond from 1 to 3 vesicles bound to a virus particle. Current camera technology now makes it possible to perform the same level of detection using the film technology illustrated in FIG. 3.

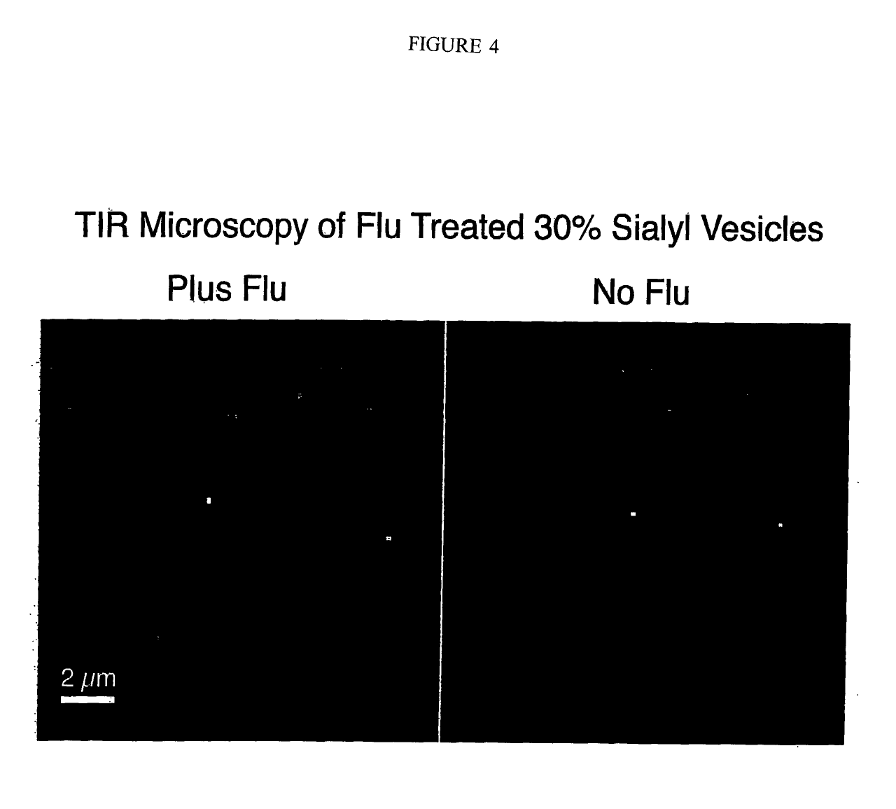

### DEFINITIONS

To facilitate an understanding of the present invention, a number of terms and phrases are defined below:

As used herein, the term “optical detector” or “photodetector” refers to a device that generates an output signal when irradiated with optical energy (e.g., fluorescence). Thus, in its broadest sense the term optical detector system is taken to mean a device for converting energy from one form to another for the purpose of measurement of a physical quantity or for information transfer. Optical detectors include but are not limited to photomultipliers and photodiodes, and charge coupled devices (CCDs).

As used herein, the term “processor” refers to a device that performs a set of steps according to a program (e.g., a digital computer). Processors, for example, include Central Processing Units (“CPUs”), electronic devices, or systems for receiving, transmitting, storing and/or manipulating digital data under programmed control.

As used herein, the term “memory device,” or “computer memory” refers to any data storage device that is readable by a computer, including, but not limited to, random access memory, hard disks, magnetic (floppy) disks, compact discs, DVDs, magnetic tape, and the like.

As used herein, the term “immobilization” refers to the attachment or entrapment, either chemically or otherwise, of a material to another entity (e.g., a solid support) in a manner that restricts the movement of the material.

As used herein, the terms “material” and “materials” refer to, in their broadest sense, any composition of matter.

As used herein, the term “polymeric material” refers to a material composed of at least one species of repeating polymer units (e.g., polydiacetylene or polythiophene). In preferred embodiments, polymeric materials can be induced to produce a fluorescent emission when binding an analyte (e.g., toxin, toxoid, antibody, virus, and the like) with affinity for the polymeric material and/or affinity to one or more ligands associated with the polymeric material. Such materials include, but are not limited to, films, vesicles, liposomes, tubules, braided assemblies, lamellar assemblies, helical assemblies, multilayers, aggregates, membranes, and solvated polymers (e.g., polydiacetylene or polythiophene aggregates such as rods and coils in solvent). Polymeric material(s) can further contain molecules that are not part of the polymerized matrix (i.e., molecules that are not polymerized). In some preferred embodiments, polymeric materials are in the form of vesicles, although the invention is not limited to this particular form of polymeric materials.

As used herein, the term “biopolymeric material” refers to materials composed of polymerized biological molecules (e.g., lipids, proteins, carbohydrates, nucleic acids, and combinations thereof). Such materials include, but are not limited to, films, vesicles, liposomes, tubules, braided assemblies, lamellar assemblies, helical assemblies, multilayers, aggregates, membranes, and solvated polymers (e.g., polydiacetylene or polythiophene aggregates such as rods and coils in solvent). Biopolymeric material(s) can further contain molecules that are not part of the polymerized matrix (i.e., molecules that are not polymerized).

As used herein, the term “biopolymeric films” refers to polymerized organic films that are used in a thin section or in a layer form. Such films can include, but are not limited to, monolayers, bilayers, and multilayers. Biopolymeric films can mimic biological cell membranes (e.g., in their ability to interact with other molecules such as proteins or analytes).

As used herein, the term “direct fluorescent detection” refers to the detection of fluorescence without or without the aid of an intervening processing step (e.g., conversion of the fluorescence into an electronic signal that is processed by an interpreting device). Thus, it is intended that the term encompass visual observing (e.g., observing with the human eye), as well as detection aided by mechanical and/or optical mechanical means.

As used herein, the term “analytes” refers to any material that is to be analyzed. Such materials can include, but are not limited to, molecules, bacteria, organic or inorganic compounds, viruses, cells, antibodies, nucleic acids, and cell parts.

As used herein, the term “selective binding” refers to the binding of one material to another in a manner dependent upon the presence of a particular molecular structure (i.e., specific binding). For example, a receptor will selectively bind ligands that contain the chemical structures complementary to the ligand binding site(s). Selective binding is contrasted to random (i.e., non-specific) binding.

As used herein, the term “biosensors” refers to any sensor device that is partially or entirely composed of biological molecules. In a traditional sense, the term refers to “an analytical tool or system consisting of an immobilized biological material (such as enzyme, antibody, whole cell, organelle, or combination thereof) in intimate contact with a suitable transducer device which will convert the biochemical signal into a quantifiable electrical signal” (Gronow, Trends Biochem. Sci., 9:336 [1984]).

As used herein, the term “transducer device” refers to a device that is capable of converting a non-electrical phenomenon into electrical information, and transmitting the information to a device that interprets the electrical signal. Such devices can include, but are not limited to, devices that use photometry, fluorometry, and chemiluminescence; fiber optics and direct optical sensing (e.g., grating coupler); surface plasmon resonance; potentiometric and amperometric electrodes; field effect transistors; piezoelectric sensing; and surface acoustic wave.

As used herein, the term “stability” refers to the ability of a material to withstand deterioration or displacement and to provide reliability and dependability.

As used herein, the term “conformational change” refers to the alteration of the molecular structure of a substance. It is intended that the term encompass the alteration of the structure of a single molecule or molecular aggregate (e.g., the change in structure of polythiophenes or glycopolythiophenes upon interaction with an analyte).

As used herein, the term “membrane” refers to, in its broadest sense, a thin sheet or layer of material. It is intended that the term encompass all “biomembranes” (i.e., any organic membrane including, but not limited to, plasma membranes, nuclear membranes, organelle membranes, and synthetic membranes). Typically, membranes are composed of lipids, proteins, glycolipids, steroids, sterols and/or other components. As used herein, the term “membrane fragment” refers to any portion or piece of a membrane. The term “polymerized membrane” refers to membranes that have undergone partial or complete polymerization.

As used herein, the term “polymerization” encompasses any process that results in the conversion of molecular monomers or structures, e.g., PDA, into larger molecules consisting of repeated monomer or structural, e.g., polymerized PDA, units. Typically, polymerization involves chemical crosslinking of monomers to one another.

As used herein, the term “membrane receptors” refers to constituents of membranes that are capable of interacting with other molecules or materials. Such constituents can include, but are not limited to, proteins, lipids, carbohydrates, and combinations thereof.

As used herein, the term “volatile organic compound” or “VOC” refers to organic compounds that are reactive (i.e., evaporate quickly, explosive, corrosive, etc.), and typically are hazardous to human health or the environment above certain concentrations. Examples of VOCs include, but are not limited to, alcohols, benzenes, toluenes, chloroforms, and cyclohexanes.

As used herein, the term “chelating compound” refers to any compound composed of or containing coordinate links that complete a closed structure.

As used herein, the term “molecular recognition complex” refers to any molecule, molecular group, or molecular complex that is capable of recognizing (i.e., specifically interacting with) a molecule.

As used herein, the term “ambient condition” refers to the conditions of the surrounding environment (e.g., the temperature of the room or outdoor environment in which an experiment occurs).

As used herein, the term “room temperature” refers, technically, to temperatures approximately between 20 and 25 degrees centigrade. However, as used generally, it refers to the any ambient temperature within a general area in which an experiment is taking place.

As used herein, the terms “home testing” and “point of care testing” refer to testing that occurs outside of a laboratory environment. Such testing can occur indoors or outdoors at, for example, a private residence, a place of business, public or private land, in a vehicle, under water, as well as at the patient's bedside.

As used herein, the term “drug” refers to a substance or substances that are used to diagnose, treat, or prevent diseases or conditions. Drugs act by altering the physiology of a living organism, tissue, cell, or in vitro system that they are exposed to. It is intended that the term encompass antimicrobials, including, but not limited to, antibacterial, antifungal, and antiviral compounds. It is also intended that the term encompass antibiotics, including naturally occurring, synthetic, and compounds produced by recombinant DNA technology.

As used herein, the term “peptide” refers to any substance composed of two or more amino acids.

As used herein the term “protein” is used in its broadest sense to refer to all molecules or molecular assemblies containing two or more amino acids. Such molecules include, but are not limited to, proteins, peptides, enzymes, antibodies, receptors, lipoproteins, glycoproteins, and transmembrane channels.

As used herein, the term “enzyme” refers to molecules or molecule aggregates that are responsible for catalyzing chemical and biological reactions. Such molecules are typically proteins, but can also comprise short peptides, RNAs, or other molecules.

As used herein the term “antibody” refers to a glycoprotein, or portion thereof, evoked in an animal by an immunogen (antigen). An antibody demonstrates specificity to the immunogen, or, more specifically, to one or more epitopes contained in the immunogen. Native antibody comprises at least two light polypeptide chains and at least two heavy polypeptide chains. Each of the heavy and light polypeptide chains contains at the amino terminal portion of the polypeptide chain a variable region (i.e., VH and VL respectively), which contains a binding domain that interacts with antigen. Each of the heavy and light polypeptide chains also comprises a constant region of the polypeptide chains (generally the carboxy terminal portion) which may mediate the binding of the immunoglobulin to host tissues or factors influencing various cells of the immune system, some phagocytic cells and the first component (C1q) of the classical complement system. The constant region of the light chains is referred to as the “CL region,” and the constant region of the heavy chain is referred to as the “CH region.” The constant region of the heavy chain comprises a CH1 region, a CH2 region, and a CH3 region. A portion of the heavy chain between the CH1 and CH2 regions is referred to as the hinge region (i.e., the “H region”). The constant region of the heavy chain of the cell surface form of an antibody further comprises a spacer-transmembranal region (M1) and a cytoplasmic region (M2) of the membrane carboxy terminus. The secreted form of an antibody generally lacks the M1 and M2 regions.

As used herein, the term “carbohydrate” refers to a class of molecules including, but not limited to, sugars, starches, cellulose, chitin, glycogen, and similar structures. Carbohydrates can also exist as components of glycolipids and glycoproteins.

As used herein, the term “lipid” refers to a variety of compounds that are characterized by their solubility in organic solvents. Such compounds include, but are not limited to, fats, waxes, steroids, sterols, glycolipids, glycosphingolipids (including gangliosides), phospholipids, terpenes, fat-soluble vitamins, prostaglandins, carotenes, and chlorophylls. As used herein, the phrase “lipid-based materials” refers to any material that contains lipids.

As used herein, the term “lipid cleavage” refers to any reaction that results in the division of a lipid or lipid-comprising material into two or more portions. “Lipid cleavage means” refers to any means of initiating and/or catalyzing lipid cleavage. Such lipid cleavage means include, but are not limited to enzymes, free radical reactions, and temperature changes.

As used herein, the phrase “free floating aggregates” refers to aggregates that are not immobilized.

As used herein, the term “encapsulate” refers to the process of encompassing, encasing, or otherwise associating two or more materials such that the encapsulated material is immobilized within or onto the encapsulating material.

As used herein, the term “optical transparency” refers to the property of matter whereby the matter is capable of transmitting light such that the light can be observed by visual light detectors (e.g., eyes and detection equipment).

As used herein, the term “biologically inert” refers to a property of material whereby the material does not chemically react with biological material.

As used herein, the term “organic solvents” refers to any organic molecules capable of dissolving another substance. Examples include, but are not limited to, chloroform, alcohols, phenols, and ethers.

As used herein, term “nanostructures” refers to microscopic structures, typically measured on a nanometer scale. Such structures include various three-dimensional assemblies, including, but not limited to, liposomes, films, multilayers, braided, lamellar, helical, tubular, and fiber-like shapes, and combinations thereof. Such structures can, in some embodiments, exist as solvated polymers in aggregate forms such as rods and coils.

As used herein, the term “films” refers to any material deposited or used in a thin section or in a layer form.

As used herein, the term “vesicle” refers to a small enclosed structures. Often the structures are membranes composed of lipids, proteins, glycolipids, steroids or other components associated with membranes. Vesicles can be naturally generated (e.g., the vesicles present in the cytoplasm of cells that transport molecules and partition specific cellular functions) or can be synthetic (e.g., liposomes).

As used herein, the term “liposome” refers to artificially produced lipid complexes (e.g., spheres, and elongated structures) that can be induced to segregate out of aqueous media (e.g., an emulsion).

As used herein, the term “biopolymeric liposomes” refers to liposomes that are composed entirely, or in part, of biopolymeric material.

As used herein, the term “tubules” refers to materials comprising small hollow cylindrical structures.

As used the term “multilayer” refers to structures comprised of two or more monolayers. The individual monolayers may chemically interact with one another (e.g., through covalent bonding, ionic interactions, van der Waals' interactions, hydrogen bonding, hydrophobic or hydrophilic assembly, and steric hindrance) to produce a film with novel properties (i.e., properties that are different from those of the monolayers alone).

As used herein, the terms “self-assembling monomers” and “lipid monomers” refer to molecules that spontaneously associate to form molecular assemblies. In one sense, this can refer to surfactant molecules that associate to form surfactant molecular assemblies. “Surfactant molecular assemblies” refers to an assembly of surface active agents that contain chemical groups with opposite polarity, form oriented monolayers at phase interfaces, form micelles (colloidal particles in aggregation colloids), and have detergent, foaming, wetting, emulsifying, and dispersing properties.

As used herein, the term “organized mono and multi-molecualr thin films” refers to polymeric materials that are mechanically assembled (e.g., on a Langmuir trough). In some embodiments, refers to layerd polymeric structures having two or more layers (e.g., bilayers, trilayers, etc.).

As used herein, the term “homopolymers” refers to materials comprised of a single type of polymerized molecular species. The phrase “mixed polymers” refers to materials comprised of two or more types of polymerize molecular species.

As used herein, the term “ligands” refers to any ion, molecule, molecular group, or other substance that binds to another entity to form a larger complex. Examples of ligands include, but are not limited to, peptides, carbohydrates, nucleic acids, antibodies, or any molecules that bind to receptors. The term “non-protein ligands” refers to all such ligands with the exception of proteins (defined above).

As used herein, the terms “organic matrix” and “biological matrix” refer to collections of organic molecules that are assembled into a larger multi-molecular structure. Such structures can include, but are not limited to, films, monolayers, and bilayers. As used herein, the term “organic monolayer” refers to a thin film comprised of a single layer of carbon-based molecules. In one embodiment, such monolayers can be comprised of polar molecules whereby the hydrophobic ends all line up at one side of the monolayer. The term “monolayer assemblies” refers to structures comprised of monolayers. The term “organic polymer matrix” refers to organic matrices whereby some or all of the molecular constituents of the matrix are polymerized.

As used herein, the phrase “head group functionality” refers to the molecular groups present an the ends of molecules (e.g., the carboxylic acid group at the end of fatty acids).

As used herein, the term “hydrophilic head-group” refers to ends of molecules that are substantially attracted to water by chemical interactions including, but not limited to, hydrogen-bonding, van der Waals' forces, ionic interactions, or covalent bonds. As used herein, the term “hydrophobic head-group” refers to ends of molecules that associate with other hydrophobic entities, resulting in their exclusion from water.

As used herein, the term “carboxylic acid head groups” refers to organic compounds containing one or more carboxyl (—COOH) groups located at, or near, the end of a molecule. The term carboxylic acid includes carboxyl groups that are either free or exist as salts or esters.

As used herein, the term “detecting head group” refers to the molecular group contained at the end of a molecule that is involved in detecting a moiety (e.g., an analyte).

As used herein, the term “linker” or “spacer molecule” refers to material that links one entity to another. In one sense, a molecule or molecular group can be a linker that is covalent attached two or more other molecules (e.g., linking a ligand or receptor to a monomer).

As used herein, the phrase “polymeric assembly surface” refers to polymeric material that provides a surface for the assembly of further material (e.g., a biopolymeric surface of a film, liposome, or polymer that provides a surface for attachment and assembly of ligands).

As used herein, the phrase “fluorescent detection element” refers to material that is capable of providing fluorescence analysis (e.g., polymerized polythiophene, or glycopolythiophene).

As used herein, the term “formation support” refers to any device or structure that provides a physical support for the production of material. In some embodiments, the formation support provides a structure for layering and/or compressing films.

As used herein, the terms “standard trough” and “standard Langmuir-Blodgett trough” refer to a device, usually made of teflon, that is used to produce Langmuir films. The device contains a reservoir that holds an aqueous solution and moveable barriers to compress film material that are layered onto the aqueous solution (See e.g., Roberts, Langmuir-Blodgett Films, Plenum, N.Y., [1990]).

As used herein, the term “domain size” refers to the typical length between domain boundaries.

As used the terms “conjugated backbone” and “polymer backbone” refer to the ene-yne polymer backbone of polymerized monomers that, on a macroscopic scale, appears in the form of physical ridges or striations. The term “polymer backbone axis” refers to an imaginary line that runs parallel to the conjugated backbone. The terms “intrabackbone” and “interbackbone” refer to the regions within a given polymer backbone and between polymer backbones, respectively. The backbones create a series of lines or “linear striations,” that extend for distances along the template surface.

As used herein, the term “bond” refers to the linkage between atoms in molecules and between ions and molecules in crystals. The term “single bond” refers to a bond with two electrons occupying the bonding orbital. Single bonds between atoms in molecular notations are represented by a single line drawn between two atoms (e.g., C₈-C₉). The term “double bond” refers to a bond that shares two electron pairs. Double bonds are stronger than single bonds and are more reactive. The term “triple bond” refers to the sharing of three electron pairs. As used herein, the term “ene-yne” refers to alternating double and triple bonds. As used herein the terms “amine bond,” “thiol bond,” and “aldehyde bond” refer to any bond formed between an amine group (i.e., a chemical group derived from ammonia by replacement of one or more of its hydrogen atoms by hydrocarbon groups), a thiol group (i.e., sulfur analogs of alcohols), and an aldehyde group (i.e., the chemical group —CHO joined directly onto another carbon atom), respectively, and another atom or molecule.

As used herein, the term “covalent bond” refers to the linkage of two atoms by the sharing of two electrons, one contributed by each of the atoms.

As used herein, the term “spectrum” refers to the distribution of light energies arranged in order of wavelength.

As used the term “visible spectrum” refers to light radiation that contains wavelengths from approximately 400 nm to approximately 750 nm.

As used herein, the term “ultraviolet irradiation” refers to exposure to radiation with wavelengths less than that of visible light (i.e., less than approximately 380 nM) but greater than that of X-rays (i.e., greater than approximately 0.1 nM). Ultraviolet radiation possesses greater energy than visible light and is therefore, more effective at inducing photochemical reactions.

As used herein, the term “solid support” refers to a solid object or surface upon which a sample is layered or attached. Solid supports include, but are not limited to, glass, metals, gels, and filter paper, among others. “Hydrophobized solid support” refers to a solid support that has been chemically treated or generated so that it attracts hydrophobic entities and repels water.

As used herein, the phrase “solid sensor platforms” refers to any solid support used for immobilizing sensor material.

As used herein, the term “film-ambient interface” refers to a film surface exposed to the ambient environment or atmosphere (i.e., not the surface that is in contact with a solid support).

As used herein, the term “formation solvent” refers to any medium, although typically a volatile organic solvent, used to solubilize and distribute material to a desired location (e.g., to a surface for producing a film or to a drying receptacle to deposit liposome material for drying).

As used herein, the term “micelle” refers to a particle of colloidal size that has a hydrophilic exterior and hydrophobic interior.

As used herein, the term “topochemical reaction” refers to reactions that occur within a specific place (e.g., within a specific portion of a molecule or a reaction that only occurs when a certain molecular configuration is present).

As used herein, the term “molding structure” refers to a solid support used as a template to design material into desired shapes and sizes.

As used herein, the terms “array” and “patterned array” refer to an arrangement of elements (i.e., entities) into a material or device. For example, combining several types of biopolymeric material with different analyte recognition groups into an analyte-detecting device, would constitute an array.

As used herein the term “interferants” refers to entities present in an analyte sample that are not the analyte to be detected and that, preferably, a detection device will not identify, or would differentiate from the analyte(s) of interest.

As used herein, the term “badge” refers to any device that is portable and can be carried or worn by an individual working in an analyte detecting environment.

As used herein, the term “device” refers to any apparatus (e.g., multi-well plates and badges) that contain biopolymeric material. The biopolymeric material may be immobilized or entrapped in the device. More than one type of biopolymeric material can be incorporated into a single device.

As used herein, the term “halogenation” refers to the process of incorporating or the degree of incorporation of halogens (i.e., the elements fluorine, chlorine, bromine, iodine and astatine) into a molecule.

As used herein, the term “aromaticity” refers to the presence of aromatic groups (i.e., six carbon rings and derivatives thereof) in a molecule.

As used herein, the phrase “water-immiscible solvents” refers to solvents that do not dissolve in water in all proportions. The phrase “water-miscible solvents” refers to solvents that dissolve in water in all proportions.

As used herein, the terms “positive,” “negative,” and “zwitterionic charge” refer to molecules or molecular groups that contain a net positive, negative, or neutral charge, respectively. Zwitterionic entities contain both positively and negatively charged atoms or groups whose charges cancel (i.e., whose net charge is 0).

As used herein, the term “small molecules” refers to any molecule with low molecular weight (i.e., less than 10,000 atomic mass units and preferably less than 5,000 atomic mass units) that binds to ligands, interacts with ligands, or interacts with biopolymeric material in a manner that creates a conformational change.

As used herein, the term “antigen” refers to any molecule or molecular group that is recognized by at least one antibody. By definition, an antigen must contain at least one epitope (i.e., the specific biochemical unit capable of being recognized by the antibody). The term “immunogen” refers to any molecule, compound, or aggregate that induces the production of antibodies. By definition, an immunogen must contain at least one epitope (i.e., the specific biochemical unit capable of causing an immune response).

As used herein, the term “biological organisms” refers to any carbon-based life forms.

As used herein, the term “pathogen” refers to disease causing organisms, microorganisms, or agents including, but not limited to, viruses, bacteria, parasites (including, but not limited to, organisms within the phyla Protozoa, Platyhelminthes, Aschelminithes, Acanthocephala, and Arthropoda), fungi, and prions.

As used herein, the term “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. “Gram negative” and “gram positive” refer to staining patterns obtained with the Gram-staining process which is well known in the art (See e.g., Finegold and Martin, Diagnostic Microbiology, 6th Ed. (1982), CV Mosby St. Louis, pp 13-15).

As used herein, the term “virus” refers to minute infectious agents, which with certain exceptions, are not observable by light microscopy, lack independent metabolism, and are able to replicate only within a living host cell. The individual particles (i.e., virions) consist of nucleic acid and a protein shell or coat; some virions also have a lipid containing membrane. The term “virus” encompasses all types of viruses, including animal, plant, phage, and other viruses.

As used herein, the term “in situ” refers to processes, events, objects, or information that are present or take place within the context of their natural environment.

As used the term “aqueous” refers to a liquid mixture predominantly containing water, among other components.

As used herein, the term “solid-state” refers to reactions involving one or more rigid or solid-like compounds.

As used herein, the term “regularly packed” refers to the periodic arrangement of molecules within a compressed film.

As used herein, the term “filtration” refers to the process of separating various constituents within a test sample from one another. In one embodiment, filtration refers to the separation of solids from liquids or gasses by the use of a membrane or medium. In alternative embodiments, the term encompasses the separation of materials based on their relative size.

As used herein, the term “sample” is used in its broadest sense. In another sense, it is meant to include a specimen or culture obtained from any source, as well as biological and environmental samples. Biological samples may be obtained from animals (including humans) and encompass fluids (e.g., blood and blood products, such as serum, plasma, and the like, saliva, lachrymal secretions, semen, urine, cerebral spinal fluid (CSF), and the like), solids (e.g., feces), tissues, and gases (e.g., blood gases). Biological samples may also comprise small molecules (e.g., nucleic acids such as DNA and RNA, PNA, lipids, polypeptides, drugs, small molecules), molecular complexes (e.g., nucleic acid hybrids, protein complexes, cell components), and reactive complexes (e.g., complexes undergoing chemical or enzymatic reactions). Environmental samples include environmental material such as surface matter, soil, water, crystals and industrial samples. These examples are not to be construed as limiting the sample types applicable to the present invention.

General Description of the Invention

The present invention relates to methods and compositions for the direct detection of analytes using the fluorescence that occurs in certain biopolymeric materials in response to the selective binding of one or more analytes to the biopolymeric materials. In particular, the present invention provides methods and compositions related to polymerized biological material that incorporate ligands (e.g., proteins) with affinity for analytes. Upon associating with the analytes (e.g., binding of the analyte to a ligand), the biopolymeric material produces a detectable fluorescence. The present invention provides for the detection of the presence of a wide range of analytes due to production of fluorescence in a biopolymeric assay material, including, but not limited to, small molecules, pathogenic and non-pathogenic organisms, toxins, membrane receptors, membrane fragments, volatile organic compounds (VOCs), enzymes, enzyme substrates, drugs, antimicrobials, antibodies, antigens, and other materials of interest.

The present invention provides analyte detecting technology that is cost-efficient, stable, accurate, reliable, consistent, robust, and very sensitive. These enhanced qualities provide an ideal basis for use in screening new compound libraries (e.g., drug screens), drug testing, fiber optic methods for remote sensing, water supply testing, and any application in which a rapid and accurate fluorescent screen is desired.

Recent research has found that liposomes and other lipid-based materials (e.g., polymeric films) can perform as sensitive optical sensors for the detection of viruses (see e.g., Reichert et al., J. Am. Chem. Soc. 117: 829 [1995]; Spevak et al., J. Am. Chem. Soc. 115: 1146 [1993]; and Charych et al., Science 261: 585 [1993]). These materials exhibit rapid response times, selectivity, and optical signals that are easily monitored. As free-floating aggregates in solution, these lipid-based detectors show promise as simple assay systems. As immobilized films, liposomes, or other forms, these detectors provide durable, robust fluorometric sensors that can be easily incorporated into small detection devices (e.g., a detection badge).

The presently claimed invention provides embodiments in which lipid based biopolymeric materials have been further refined to produce fluorescing assay materials. Some embodiments of the present invention incorporate one or more ligands, such as peptides, proteins, and antibodies. The present invention contemplates that these ligands allow for the fluorescent detection of a broad range of analytes (e.g. ions, carbohydrates, proteins, lipids, and antigens), while also providing specificity by detecting the interaction between individual ligand types and only one or a small class of analytes. The present invention further contemplates biopolymeric materials containing multiple ligands for the detection of larger classes of analytes. The present invention also contemplates an array of biopolymeric materials incorporated into a single device, such that each individual section of biopolymeric material responds to a different analyte. Such arrays can be designed so that the presence of a given analyte will produce fluorescence in a known location in the device, or that will produce a fluorescence specific to the given analyte. It is also contemplated that other arrays will be used with the present invention, including such easily understood patterns as a “+” sign to indicate that presence of a particular substance or compound. It is not intended that the present invention be limited to any particular array design or configuration.

Thus, the present invention provides methods and compositions that fulfill many of the goals of the analyte detection field and overcomes many of the disadvantages of currently available technologies. The present invention provides significant advantages over previously used biosensors, as the embodiments of the present invention are extremely sensitive.

### DETAILED DESCRIPTION OF THE INVENTION

The present invention relates to methods and compositions for the detection of analytes using the fluorescence that occurs in biopolymeric and/or polymeric materials in response to selective binding of analytes to the materials. In particular, the present invention allows for the fluorescent detection of membrane modifying reactions and analytes responsible for such modifications and for the screening of reaction inhibitors.

In preferred embodiments, the fluorescence that is induced upon bind of an analyte (e.g., virus) to the materials of the present invention is detected by a suitable device (e.g., spectrofluorometer). Colorimetric analyte detection methods are based on measuring changes in light absorbance, whereas fluorogenic sensor are based upon measuring the conversion of a non-fluorescent form of a material (e.g., the biopolymeric or polymeric materials disclosed herein) into a fluorescent light emitting material. Accordingly, it is contemplated that the present fluorogenic methods disclosed herein are considerably more sensitive than existing colorimetric methods. For example, in preferred embodiments, fluorescence detection methods are capable of detecting the emission of single photos of light and to collect the whole emission spectra of single fluor molecules. In contrast, colorimetric techniques maximally optimized, depend upon measuring small differences between large signals which inherently contain an amount of noise. As a practical example, a standard high quality spectrophotometer can measure about 1 micromolar of fluorescein dye with a few percent of error. While a standard spectrofluorometer can detect a concentration of about 50 femtomolar of fluorescein dye. Preferred embodiments of the present invention, are directed to methods of detecting the fluorescent emissions from suitable polymeric and/or biopolymeric materials.

Preferred embodiments of the present invention are based upon the inventor's discovery of the intense and stable fluorescence produced in polydiacetylene films in discrete domains when activated by side chain crosslinked silalyl headgroups when bound to virus particles (e.g., influenza virions). Preferred films are non-fluorescent except when bound to a suitable activating analyte (e.g., a pathogen or drug). It is contemplated that this greatly increases the signal-to-noise ratio of the methods and systems based thereon as compared to standard colorimetric assays methods and systems and also increases the rate of detection of the present methods by several orders of magnitude.

Experimental testing using high resolution CDD, visible light microscopy, and total internal reflection microscopy (TIR) have shown that preferred embodiments the present invention provide ultra sensitive and ultra-rapid detection of single virus particles and potentially any other pathogen with receptors that can be derivatized to bind to the present polydiacetylene (PDA) surface headgroups. Conversely, in other embodiments, the polydiacetylene (PDA) surface headgroups are suitable derivatized to bind analytes of interest.

In still other embodiments, the general fluorescence response of the disclosed PDA films is used to make arrays of specific domains that allow sampling of multiple ligands in a single sample. For instance, experimental results indicate that a 100×100 μm array can be used be create a detector with a minimum dynamic range of 14 bits (0-16,000) on up to a range of 17 bits (0-128,000). Moreover, an 10×10 array of these 1,000 sq. μm domains, easily manufactured by molecular film deposition techniques, can evaluate 100 different analytes (e.g., pathogens or chemical species) at a time. Thus, in particularly preferred embodiments, one 2.5×7.5 cm glass slide, can be used to create an array of 10,000 domains of 100×100 μm arrays for pathogen or drug testing and screening, etc.

These biopolymeric and/or polymeric materials comprise many forms including, but not limited to, films, vesicles, liposomes, tubules, and multilayered structures. These materials are comprised of polymerized monomers. In some embodiments, the biopolymeric materials comprise more than one species of monomer. Some of these monomers may lack polymerizable groups. In other embodiments, the materials further comprise dopant material(s) that alter the properties of the sensor. Dopants include, but are not limited to, polymerizable monomers, non-polymerizable monomers, lipids, cholesterols, membrane components and any other molecule that optimizes the biopolymeric material (e.g., material stability, durability, fluorescent response, and immobilizability). The biopolymeric material may further comprise ligand(s). The ligands provide recognition sites for analytes, such that binding of the analyte to the ligand results in a fluorescent change of the biopolymeric material. The use of particular ligands can be used to selectively detect the presence of a specific analyte(s). The various embodiments of the presently claimed invention provide the ability to fluorometrically detect a broad range of analytes. The analytes either interact directly with the monomers or with ligands that are linked to or associated with the monomers. The biopolymeric materials fluoresce upon binding an analyte. The present invention provides a variety of means of immobilizing the biopolymeric material to provide stability, durability, and ease of handling and use. In some embodiments, a variety of different polymeric materials are combined into a single device to produce an array. The array can be designed to detect and differentiate differing types or quantities of analytes (i.e., the array can provide quantitative and/or qualitative data). The methods and compositions of the presently claimed invention find use in a broad range of analyte detection circumstances and are particularly amenable to situations where very sensitive analyte detection is required.

The description of the invention is divided into: I. Forms of Biopolymeric Materials; II. Self-Assembling Monomers; III. Dopants; IV. Ligands; V. Detection of Analytes; VI. Immobilization of Biopolymeric Materials; VII. Arrays; and VII. Total Internal Reflection Microscopy.

I. Forms of Biopolymeric Materials

The biopolymeric material of the presently invention can take many physical forms including, but not limited to, liposomes, films, and multilayers, as well as braided, lamellar, helical, tubular, and fiber-like shapes, and combinations thereof. Each of these classes is described below, highlighting their advantages and the difficulties overcome during the development of these materials.

A. Films

In some embodiments, the biopolymeric material used in the presently claimed invention comprises biopolymeric films. Films include monolayers, bilayers, and multilayers. As described in Example 1, biopolymeric films are prepared by layering the desired matrix-forming material (e.g., self-assembling organic monomers) onto a formation support. In preferred embodiments, the formation support is a standard Langmuir-Blodgett trough and the matrix-forming material is layered onto an aqueous surface created by filling the trough with an aqueous solution. The material is then compressed and polymerized to form a biopolymeric film. In preferred embodiments, the compression is conducted in a standard Langmuir-Blodgett trough using moveable barriers to compress the matrix-forming material. Compression is carried out until a tight-packed layer of the matrix-forming material is formed. Films provide a very sensitive fluorescent screen for analytes.

As described in Example 1, in some embodiments, the matrix-forming material, located within the formation support, is polymerized by ultra-violet irradiation. All methods of polymerization are contemplated by the present invention and include, but are not limited to, gamma irradiation, x-ray irradiation, and electron beam exposure.

In some embodiments, lipids comprising diacetylene monomers (DA) are used as the self-assembling monomer. The diacetylene lipid monomers (DA) are polymerized to polydiacetylene (p-PDA or PDA) using ultraviolet irradiation. In preferred embodiments, the ultraviolet radiation source is kept sufficiently far from the film to avoid causing heat damage to the film. The crystalline morphology of the polymerized film can be readily observed between crossed polarizers in an optical microscope, although this step is not required by the present invention.

In certain embodiments the films are then transferred to hydrophobized solid supports, such that the carboxylic acid head groups are exposed at the film-ambient interface (Charych et al., Science 261: 585 [1993]) to undergo further analysis, although the method of the present invention does not require this step. Linear striations of the PDA films can be observed in the polarizing optical microscope. The material may also be characterized using atomic force microscopy or other characterization means (See, Example 2).

B. Liposomes

In other embodiments, the biopolymeric materials used in the presently claimed invention comprises biopolymeric liposomes (i.e., vesicles). In some embodiments, liposomes are prepared using a probe sonication method (New, Liposomes: A Practical Approach, Oxford University Press, Oxford, pp 33-104 [1990]), although any method that generates liposomes is contemplated. Self-assembling monomers, either alone, or associated with a desired ligand, are dried to remove the formation solvents and resuspended in deionized water. The suspension was probe sonicated and polymerized. The resulting liposome solution contained biopolymeric liposomes.

Liposomes differ from monolayers and films in both their physical characteristics and in the methods required to generate them. Monolayers and films (or multilayers) made from amphiphilic compounds are planar membranes and form a two-dimensional architecture. Monolayers and films, in this context, are solid state materials that are supported by an underlying solid substrate. Such materials are described in numerous articles and have been reviewed in texts such as Ulman (Ulman, An Introduction to Ultrathin Organic Films: From Langmuir-Blodgett to Self-Assembly, Academic Press, Inc., Boston, [1991]) and Gaines (Gaines, Insoluble Monolayers at Liquid-Gas Interfaces, Interscience Publishers, New York, [1966]). In contrast to films and monolayers, liposomes are three-dimensional vesicles that enclose an aqueous space. These materials are described in numerous articles and have been reviewed in texts such as New (New, Liposomes: A Practical Approach, IRL Press, Oxford, [1989]), and Rosoff (Rosoff, Vesicles, Marcel Dekker, Inc., New York, [1996]) among others. Liposomes can be constructed so that they entrap materials within their aqueous compartments. Films and monolayers do not enclose an aqueous space.

Liposomes and films are prepared using different methods. Liposomes are prepared by dispersal of amphiphilic molecules in an aqueous media and remain in the liquid phase. In contrast, monolayers and films are prepared by immobilizing amphiphilic molecules at an air-water interface. A solid support is then passed through the interface to transfer the film to the solid support. Liposomes exist within homogenous aqueous suspensions and may be created in a variety of shapes such as spheres, ellipsoids, squares, rectangles, and tubules. Thus, the surface of a liposome is in contact with liquid only—primarily water. In some respects, liposomes resemble the three-dimensional architecture of natural cell membranes. If liposomes are dried to their solid state, they may lose their shape and no longer exist in a liposomal state (i.e., are no longer “liposomes”). In contrast, films exist as planar heterogeneous coatings, immobilized onto a solid support. The surface of a monolayer or film can be in contact with air, other gases, or other liquids. Films can be dried in air and maintain their planar monolayer or multilayer structure and thus remain as “films.”

A much higher concentration of polymerized material can be achieved in polymerized assemblies with liposome solutions compared to monolayer assemblies, due to their greater cross-sectional density. In some embodiments, liposome compositions are fabricated using supercooling techniques described in the art.

C. Other Forms

In other embodiments, it is contemplated that variations in the heating and cooling rates, agitation methods, and materials of the biopolymeric material will provide other nanostructures. Such nanostructures include, but are not limited to, multilayers, braided, lamellar, helical, tubular, and fiber-like shapes, and combinations thereof. Such structures can, in some embodiments, be solvated polymers in aggregate forms such as rods and coils. It has been shown that the chain length of the monomers effects the type of aggregate that forms in solution (Okahata and Kunitake, J. Am. Chem. Soc. 101: 5231 [1979]). Generation of these other forms with surfactant materials has been described for double chains (Kuo et al., Macromolecule 23: 3225 [1990]), lamellac (Rhodes et al., Langmuir 10: 267 [1994]), and hollow tubules and braids (Frankel et al., J. Am. Chem. Soc. 116 [1994]). In some embodiments, fluorometric tubules are generated. As described in Example 1, tubules are prepared similarly to liposomes, except that 1-10% of an organic solvent (e.g., ethanol) is added to the solution prior to sonication. The present invention also contemplates other shapes suitable for particular uses as desired.

II. Self-Assembling Monomers

In certain embodiments, the present invention contemplates a variety of self-assembling monomers that are suitable for formation of biopolymeric materials. In some embodiments, such monomers include, but are not limited to, acetylenes, diacetylenes (e.g., 5,7-docosadiynoic acid, 5,7-pentacosadiynoic acid, and 10,12-pentacosadiynoic acid), alkenes, thiophenes, polythiophenes, imides, acrylamides, methacrylates, vinylether, malic anhydride, urethanes, allylamines, siloxanes, anilines, pyrroles, and vinylpyridinium. Lipids containing these groups can be homopolymers or mixed polymers. Furthermore, monomers with a variety of head groups are contemplated, including, but not limited to carboxylic acid, hydroxyl groups, primary amine functionalities, amino acid derivatives, and hydrophobic groups. Certain head groups may act as recognition sites for binding to analytes, allowing direct fluorometric detection, simply through exposure of the biopolymeric material to the analyte. In preferred embodiments the head groups are hydrophilic. In particularly preferred embodiments, the hydrophilic head groups further comprise lipids.

The biopolymeric material of the present invention may comprise a single species of self-assembling monomer (e.g., may be made entirely of 5,7-pentacosadiynoic acid) or may comprise two or more species. To produce biopolymeric material with more than one type of self-assembling monomer, solvents containing the individual monomers are combined in the desired molar ratio. This mixture is then prepared as described above (e.g., layering onto the aqueous surface of a Langmuir-Blodgett device for film preparation or evaporated and resuspended in aqueous solution for liposome preparation). In some embodiments, the monomers (and in some cases self-associated monomers) may be chemically linked to another molecule (e.g., a ligand).

In preferred embodiments, lipid monomers comprising diacetylene are used as the self-assembling monomers of the biopolymeric material of the present invention. Various embodiments of the present invention contemplate a variety of diacetylene-containing lipids including, but not limited to 5,7-docosadiynoic acid (5,7-DCDA), 5,7-pentacosadiynoic acid (5,7-PCA), and 10,12-pentacosadiynoic acid (10,12-PCA).

The presently claimed invention further contemplates the optimization of the biopolymeric material to maximize response to given analytes. Although it is not necessary to understand the mechanism in order to use the present invention, and it is not intended that the present invention be so limited, it is contemplated that the chemistry of the particular lipid used in the biopolymeric material plays a critical role in increasing or decreasing the sensitivity of the fluorometric transition. For example, a positional variation of the polymer backbone can alter sensitivity to a given analyte. This may be accomplished by moving the diacetylene group closer to the interfacial region (e.g., 5,7-pentacosadiynoic acid as opposed to 10,12-pentacosadiynoic acid). In addition, shorter or longer chain lengths of PDA are predicted to have an effect on the analyte permeation due to changes in packing. It is contemplated that improved sensitivity allows better detection of small analytes (e.g., bacterial toxins such as cholera toxin from Vibrio cholerae and pertussis toxin, as well as antibodies). It is also contemplated that optimization will allow sensitive detection of many large, low valence analytes, weak binding analytes, and analytes present at even very low concentrations.

A. Polymerizable Group Placement in Monomer Carbon Chain

The carbon chain length that positions the head group at a specific distance from the polymer backbone in the final polymerized material is dependent on the position of the polymerizable group in an unassembled monomer. In the case of diacetylene liposomes, some embodiments of the present invention contemplate that a diacetylene group positioned from between the 18-20 positions to the 3-5 position in the monomers will produce progressively more sensitive liposomes. Furthermore, the present invention contemplates that liposomes produced from monomers with the diacetylene groups from the 10-12 position to the 4-6 position will provide particularly efficient control of sensitivity. Diacetylene groups positioned in about the 5-7 position are preferred for certain embodiments. The production protocol for the monomer determines at which position the diacetylene group is placed in the final monomer product.

B. Total Carbon Chain Length

Experiments of the present invention demonstrated that the total carbon chain length in the unassembled monomer also influenced the level of sensitivity of the liposome product, although to a lesser extent than the position of the polymerizable group in the monomer carbon chain. The shorter chain length typically provided for greater sensitivity. The monomers that are ideally useful in construction of the inventive fluorometric liposomes range from between C₁₂ to C₂₅ in length, although both longer and shorter chain lengths are contemplated by the presently claimed invention. A preferred range of monomer carbon chain length in the present invention is C₂₀ to C₂₃.

C. Polydiacetylene Lipids and Derivatives

In search of ideal biopolymeric systems that are capable of providing fluorescent signal transduction and the mechanical stability to host recognition units in biosensors, a series of diacetylenic lipids with various headgroups are synthesized. The approach also reflects the efforts in looking for powerful dopants in the mixed matrix that readily bring the sensor film/liposomes into “meta-stable state” as to promptly respond to any occurring binding events at the interface. The present invention provides PDA whereby the carboxylate headgroup has been converted to isoleucine, glycine, serine, cystine, glutamic acid, histidine, phenylalanine, arginine, glutamine, pentaethalenehexamine (PEH), 3-dimethylaminopropylamine (DMAP), N-hydroxysuccinimide (NHS). It is also contemplated that the present invention include functionalizing diacetylenic lipids including peptides-PDA, oligo-PDA, chelate-PDA, fluorescence-PDA, and antibody-PDA.

In some embodiments, the present invention provided for the charge-induced fluorescent transition of amino acid terminated liposomes containing polydiacetylene moiety. Amphiphilic amino acids are synthesized according to established synthetic route, and the liposomes are prepared using sonication. This procedure allows examination of liposome formation of lipids with varied headgroups.

III. Dopants

In some embodiments, the use of promoter molecules to dope the fluorometric assay liposomes of the present invention provided a dramatic intensity of fluorescence at low levels of analyte, allowing unprecedented sensitivity and improved quantitation of analyte detection.

A. Dopant Materials

The biopolymeric materials of the present invention may further comprise one or more dopant materials. Dopants are included to alter and optimize desired properties of the biopolymeric materials. Such properties include, but are not limited to, fluorometric response, sensitivity, durability, robustness, amenability to immobilization, temperature sensitivity, and pH sensitivity. Dopant materials include, but are not limited to, lipids, cholesterols, steroids, ergosterols, polyethylene glycols, proteins, peptides, or any other molecule (e.g., surfactants, polyethylene glycol, polysorbate, octoxynol, sodium dodecyl sulfate, zwitterionic detergents, decylglucoside, deoxycholate, diacetylene derivatives, phosphatidylserine, phosphatidylinositol, phosphatidylethanolamine, phosphatidylcholine, phosphatidylglycerol, phosphatidic acid, phosphatidylmethanol, cardiolipin, ceramide, cerebroside, lysophosphatidylcholine, D-erythroshingosine, sphingomyelin, dodecyl phosphocholine, N-biotinyl phosphatidylethanolamine, and other synthetic or natural components of cell membranes) that can be associated with a membrane (e.g., liposomes and films).

Different dopant materials can be combined in a single biopolymeric material preparation. For example, the present invention contemplates a dopant cocktail that is a mix of glucose and sialic acid-derived polydiacetylene. The glucose component of the dopant acts primarily to prevent non-specific adhesion to the surface of the inventive liposome and may also enhance sensitivity. The polydiacetylene bound sialic acid component would functionally destabilize the surface to provide a dramatic increase in sensitivity. By using this co-dopant approach, both specificity of adhesion and sensitivity can be optimized, without unduly compromising the structural integrity of the biopolymeric material.

In one embodiment of the present invention, a dopant constituent comprises a diacetylene lipid with a charged head group. The head group can be chosen at a larger size commensurate with the size of the target analyte. Similarly, when there is a longer chain length, the dopant head group can be of a larger size. While one would generally select the size of the head group of a diacetylene dopant constituent to be proportionate to the size of the target analyte, or of the same size as the ligand, there is also the opportunity to provide useful stearic hindrance. For instance, a larger head group on the dopant may demand very specific, rigorous binding of analyte in order to overcome stearic hindrance to analyte binding provided by the dopant. This approach would assure that non-specific binding would be limited or precluded, and represents an opportunity to improve the specificity of the inventive analytic system.

In certain embodiments, the dopant comprises a diacetylene or a modified diacetylene (e.g., sialic acid derived diacetylene). It should be noted that in this case, the derivatized lipid is used to modify the properties of the biopolymeric material and is not used as a molecular recognition site for an analyte detection (e.g., as in the case of sialic acid ligand used to detect influenza virus).

It is contemplated that a wide variety of dopant materials will find use in optimizing the properties of the biopolymeric material used in various embodiments of the present invention. Materials that are constituents of cell membrane structures in nature are generally useful as dopants in the present invention. For instance, steroids (e.g., cholesterols) represent potential dopants that can provide desired degrees of destabilization or stabilization to the biopolymeric material. Surfactant type compounds also may serve as dopants, whether or not they are polymerized to the monomers (in some cases self-assembling monomers) that make up the polymer back bone. An alternative surfactant that can be used as dopants are peptide-detergents (i.e., small amphipathic molecules that have a hydrophobic region mimicking the membrane spanning regions of membrane proteins). These small peptides (typically 20-25 amino acids in length) can be incorporated into the biopolymeric material to alter the stability or sensitivity of the fluorometric response of the material when exposed to specific analytes. In one embodiment of the present invention, lipids from natural sources are used as dopants. Excellent sources for these lipids are various yeast species, and also plant sources, such as soybeans.

Although it is not necessary to understand the mechanism in order to use the present invention, and it is not intended that the present invention be so limited, it is contemplated that the addition of dopant will lower the activation barrier of the fluorescent response and/or provides a connection between the ligands (i.e., if ligands are present) and the conjugated backbone, enabling the analyte to induce the fluorescent response.

B. Percentages of Dopants

The most appropriate percentage of dopant incorporated into the structure of the biopolymeric material is dependent on the particular analytic system being developed, and the needs of the testing situation. For instance, sensitivity may be compromised to some extent in the favor of long shelf life, or to accommodate rigorous field conditions. The acceptable percentage of dopant is theoretically limited only to that which will not preclude sufficient incorporation of the indicator polydiacetylene molecules to produce the necessary optical density and fluorescent response or to that which will disrupt the stability of the polymeric structures.

Molar percentages of dopant can vary from as low as 0.01% to as high as 75%, after which the structural integrity of the biopolymeric material typically begins to deteriorate. However, there may be specific embodiments where the percentage of dopant is greater than 75% or lower than 0.01%. A preferred range for dopant is 2%-10%. In certain embodiments of the present invention, the optimal percentage of dopant is about 5% (See e.g., Example 4, section II).

C. Dopant Incorporation Parameters

In selecting appropriate incorporation methods for the dopant, there are several competing considerations. For example, for the sonication bath method for production of certain liposome embodiments, the incorporation is very controlled, and requires several hours of processing. This relatively slow, gentle incorporation method allows the incorporation of comparatively large or complex dopant materials. However, the sonication bath approach is only suitable when it is intended that a relatively low percentage of dopant is to be incorporated. The point probe method allows the incorporation of a much higher percentage of dopant material over a shorter period of time, typically from one to ten minutes. However, this method is typically limited to incorporation of small to intermediate sized dopant materials.

The temperature chosen for incorporation are selected based on the particular analytical system and liposome parameters desired. In one embodiment, the temperatures range from 46° C. to 55° C. In another embodiment, temperatures of the bath system range from 10° C. to 30° C. In certain embodiments, sonication is accomplished at about 20° C. The temperature can be varied throughout a run to optimize results, for instance lowering the temperature to stabilize the system towards the end of the run, prior to a possible structural compromise of a large dopant material.

Furthermore, a practitioner will be able to select parameters such as pH, choice of diluents, and other factors based on the particular system and desired characteristics of the biopolymeric material.

D. Non-Synthetic Synthesis Route

In some embodiments, the present invention provides ligands that are not covalently linked to the monomers (i.e., a non-synthetic synthesis route). The non-synthetic approach to incorporating ligands to biopolymeric materials has advantages over the synthetic incorporation route since the covalent attachment of receptor molecules onto the polymer backbone can be difficult for some types of ligands (e.g., ligands with membrane spanning regions). In some embodiments employing the non-synthetic approach to incorporating ligands, dopants are also used to increase fluorometric response.

In some embodiments, dopants are provided to increase the fluorometric response of the biopolymeric assay materials. These dopants are not the same as filler molecules typically observed in biological membranes (i.e., cholesterol, proteins, lipids, detergents). They differ in that they provide unique and specific functionality to a given sensor system. For example, toxin detecting embodiments of the present invention (e.g., the detection of E. coli toxins) are preferably designed by a non-synthetic approach. The design of several dopants that provide specific functionality to the non-synthetic embodiments are described below and in Example 4.

A number of simple system can be designed so that the PDA molecule can easily be derivatized. For example, 10,12-pentacosadiynoic acid can be modified to amine-couple to any molecule with a free amino group. Since all amino acids have a free amino group (lysine has 2 free amino groups), the 20 amino acids can each be placed on the head of a PDA molecule. These amine-coupled PDAs are used to add amino acid groups into the PDA lipid matrix, or to attach proteins (i.e., by surface lysine residues) to the surface of a PDA film or liposome. Each one of the derivatized PDA molecules has special properties that allow specific functionality to be incorporated into the biopolymeric material. Each time a new sensor system is designed, the amount of PDA, derivatized PDA, and receptor molecule (i.e., ganglioside for specific toxins) are varied to create the optimal sensor.

In one embodiment of the present invention, PDA is derivatized with hydrophobic amino acid residues (i.e., Phe, Try, Try, Ile). Hydrophobic residues linked to diacetylenes lowered the solubility of PDA as well as the stability of the films/liposomes. These derivatized PDA find use in the assembly of complex PDA systems to fine tune the stability and sensitivity, two factors that are directly related to one another. For example, hydrophobic PDAs may be used with the hydrophilic PDAs, increasing the stability of biopolymeric assemblies, under a variety of environmental conditions. Although a large gain in stability is seen, it is at a cost to sensitivity. A balance between sensitivity and stability is optimized.

In another embodiment of the present invention, PDA is derivatized with acidic and basic amino acid residues (i.e., Glu, Gln, Arg, Ser). Acidic and basic residues linked to diacetylenes increased the solubility of the derivatized PDA; in particular, these changes allowed polydiacetylene lipids to mix with water-soluble biological molecules. Ordinarily, PDA is not water soluble and organic solvents are necessary (which can be destructive to biological molecules). By placing acidic or basic head groups onto the PDA molecule, the solubility of the derivatized PDAs are greatly enhanced. In some embodiments of the present invention, the biopolymeric materials are derivatized directly with fluorescent head groups.

IV. Ligands

The biopolymeric materials of the present invention may further comprise one or more ligands. Ligands act as a recognition site in the biopolymeric materials for analytes. While not limited to any particular mechanism, the present invention contemplates that upon interaction of the analyte with the ligand or ligands, a disruption of the polymer backbone of the biopolymeric material occurs, resulting in a detectable fluorescent response. In some embodiments, ligands are linked by a linking arm to the monomers (in some cases self-assembling monomers), directly linked to the monomers, incorporated into the biopolymeric matrix prior to or during the polymerization process, or attached to the matrix following polymerization (e.g., by linking ligands to matrix constituents that contain head groups capable of binding ligands, or through other means). The present invention, also contemplates non-synthetic methods for incorporating or associating ligands to biopolymeric material, whereby the ligand is not covalently linked to the polymerized matrix.

A wide variety of materials are available for use as ligands with the biopolymeric assay materials of the present invention. The main criterion is that the ligand have an affinity for the analyte of choice. Appropriate ligands include, but are not limited to, peptides, carbohydrates, nucleic acids, biotin, drugs, chromophores, antigens, chelating compounds, molecular recognition complexes, ionic groups, polymerizable groups, dinitrophenols, linker groups, electron donor or acceptor groups, hydrophobic groups, hydrophilic groups, antibodies, or any organic molecules that bind to receptors. In some embodiments, the biopolymeric material comprises combinations of ligand-linked and unlinked monomers to optimize the desired fluorometric response (e.g., 5% ligand-linked dicosadynoic acid [DCDA] and 95% DCDA). Additionally, multiple ligands can be incorporated into a single biopolymeric matrix. As is clear from the broad range of ligands that can be used with the present invention, an extremely diverse group of analytes can be detected.

In some embodiments, the monomers are not associated with ligands, but are directly assembled, polymerized, and used as fluorometric sensors. Such biopolymeric materials find use in the detection of certain classes of analytes including, but not limited to, volatile organic compounds (VOCs).

In some embodiments of the present invention, ligands directly bind to, and to detect analytes. For example, in some embodiments sialic acid derivatives are contemplated to detect influenza virus, G_{M1} gangliosides to detect cholera toxin, and G_{T1b} gangliosides to detect botulinum neurotoxin. In alternative embodiments, ligands are used in the present invention when they function as competitive binders to the analyte.

In one preferred embodiment of the present invention, ganglioside acid derivatives (i.e., a receptor-binding molecule) are used as ligands. Receptor-binding molecules are materials on the surface of a host cell to which a pathogen attaches itself as a prelude to the ineffective event. Selecting these molecules as the ligand group in the present invention provides many advantages over other receptor molecules. The recognition site for these molecules tend to be highly genetically conserved in the pathogen because of its obvious criticality to survival. Therefore, different strains of the same pathogen will generally not produce a false negative when such molecules are selected as the ligand group in the subject invention. Also, receptor molecules tend to be smaller and less complex than antibodies to the same analyte. An increasing number of receptor molecules are being recognized, identified, isolated, and synthesized for a large number of pathogens.

In some embodiments, ligands are incorporated to detect a variety of pathogenic organisms including, but not limited to, sialic acid to detect HIV (Wies et al., Nature 333: 426 [1988]), influenza (White et al., Cell 56: 725 [1989]), Chlamydia (Infect. Imm. 57: 2378 [1989]), Neisseria meningitidis, Streptococcus suis, Salmonella, mumps, newcastle, and various viruses, including reovirus, Sendai virus, and myxovirus; and 9-OAC sialic acid to detect coronavirus, encephalomyelitis virus, and rotavirus; non-sialic acid glycoproteins to detect cytomegalovirus (Virology 176: 337 [1990]) and measles virus (Virology 172: 386 [1989]); CD4 (Khatzman et al., Nature 312: 763 [1985]), vasoactive intestinal peptide (Sacerdote et al., J. of Neuroscience Research 18: 102 [1987]), and peptide T (Ruff et al., FEBS Letters 211: 17 [1987]) to detect HIV; epidermal growth factor to detect vaccinia (Epstein et al., Nature 318: 663 [1985]); acetylcholine receptor to detect rabies (Lentz et al., Science 215: 182 [1982]); Cd3 complement receptor to detect Epstein-Barr virus (Carel et al., J. Biol. Chem. 265: 12293 [1990]); β-adrenergic receptor to detect reovirus (Co et al., Proc. Natl. Acad. Sci. 82: 1494 [1985]); ICAM-1 (Marlin et al., Nature 344: 70 [1990]), N-CAM, and myelin-associated glycoprotein MAb (Shephey et al., Proc. Natl. Acad. Sci. 85: 7743 [1988]) to detect rhinovirus; polio virus receptor to detect polio virus (Mendelsohn et al., Cell 56: 855 [1989]); fibroblast growth factor receptor to detect herpes virus (Kaner et al., Science 248: 1410 [1990]); oligomannose to detect Escherichia coli; ganglioside G_{M1} to detect Neisseria meningitidis; and antibodies to detect a broad variety of pathogens (e.g., Neisseria gonorrhoeae, V. vulnificus, V. parahaemolyticus, V. cholerae, and V. alginolyticus).

Methods of derivatizing lipids with a diverse range of compounds (e.g., carbohydrates, proteins, nucleic acids, and other chemical groups) are well known in the art. The carboxylic acid on the terminal end of lipids are easily modified to form esters, phosphate esters, amino groups, ammoniums, hydrazines, polyethylene oxides, amides, and many other compounds. These chemical groups provide linking groups for carbohydrates, proteins, nucleic acids, and other chemical groups (e.g., carboxylic acids can be directly linked to proteins by making the activated ester, followed by reaction to free amine groups on a protein to form an amide linkage). Examples of antibodies attached to Langmuir films are known in the art (See e.g., Tronin et al., Langmuir 11: 385 [1995]; and Vikholm et al., Langmuir 12: 3276 [1996]). There are numerous other means to couple materials to membranes, or incorporate materials within a membrane, including for example, coupling of proteins or nucleic acids to polymer membranes (See e.g., Bamford et al. Adv. Mat. 6: 550 [1994]); coupling of proteins to selfassembled organic monolayers (See e.g., Wilner et al., Adv. Mat. 5: 912 [1993]), and incorporating proteins into membranes (See e.g., Downer et al., Biosensor and Bioelect. 7: 429 [1992]); among others. Protocols for attaching ligands (e.g., proteins, nucleic acids, and carbohydrates) to the materials of the present invention are provided in Example 5.

### Protein Ligands

The methods of the present invention provide a system to easily attach protein molecules, including antibodies, to the surface of polydiacetylene thin films and liposomes, thereby providing fluorescent biopolymeric materials with “protein” ligands. Such ligands include, but are not limited to, peptides, proteins, lipoproteins, glycoproteins, enzymes, receptors, channels, and antibodies. Upon binding an analyte (e.g., enzyme substrate, receptor ligand, antigen, and other protein), a disruption of the polymer backbone of the biopolymeric material occurs, resulting in a detectable fluorescent response. In some embodiments, protein ligands are incorporated into the biopolymeric material, while in other embodiments, protein ligands are chemically associated with the surface of the biopolymeric material (e.g., chemically linked to the surface head group of one or more monomers in the biopolymeric monomer).

Specific applications of the presently claimed invention are described below to illustrate the broad applicability of the invention to a range of analyte detection systems and to demonstrate its specificity, and ease of use. The examples presented herein are intended to merely illustrate the broad applicability of the present invention. It is not intended that the present invention be limited to any particular embodiment.

1. Antibody Ligands

The present invention contemplates other protein ligand embodiments, including those specifically designed for antibody-antigen recognition. In these embodiments, polyclonal or monoclonal antibodies are coupled to the PDA assembly surface. The stress caused by binding different antibodies to different epitopes is sufficient to induce a fluorescent response. The present invention contemplates two examples of such systems: the alpha-factor receptor (i.e., a G-protein coupled receptor) and a sensor for Chlamydia trachomatis (i.e., the causative agent of a common STD). As these examples illustrate, the attachment of antibodies to the inventive biopolymeric material provides a fluorescent detection system for an enormous range of analytes (i.e., any analyte for which an antibody can be generated).

Alpha factor receptor antibodies can be used to investigate the general procedure of immobilizing protein onto the monolayer and liposome surface to make immunochemical materials. Alpha-factor receptor antibodies are immobilized to monolayer and vesicles through amide linkage. The regular diacetylene lipid is first converted to NHS-PDA lipid and then mixed with matrix lipids to form monolayer or vesicles (e.g., liposomes), depending on the system under development. The surfaces of antibody molecules have a large number of exposed lysine residues where free amines serve as the attack agent to replace NHS on the monolayer/liposome surface to form amide bonds.

In some embodiments, it is possible to confirm that the films/liposomes are binding the protein molecules, by running the subject protein on nitrocellulose and staining with the biopolymeric liposomes, the protein molecules are detected. This detection method is very similar to Western blot analysis, without the need of additional antibodies, dyes, or other detection components. It is contemplated that this detection method will find use in the immunochemical staining arts as an addition or alternative to traditional Western blot techniques (See e.g., Example 10).

An additional embodiment provides a fluorometric assay for the detection of the Chlamydia trachomatis. Suitable polyclonal antibodies directed against the organism are obtained and immobilized onto diacetylene thin films and liposome surfaces via a similar method described above for antibody attachment through amine coupling. In this case, the target antigen is present on a bacteria (i.e., a much larger entity than the small molecule or protein antigens used previously).

Although it is not necessary to understand the mechanism in order to use the present invention, and it is not intended that the present invention be so limited, it is contemplated that with the toxin/virus sensor, the molecules directly interact with the membrane and in the hexokinase system, a gross conformational change occurs upon binding ligand. In both of these cases, there are conformational changes occurring at the membrane surface. With the antibody approach, the primary driving force for the fluorescent response is the molecule recognition of the antibody-antigen binding event. It is contemplated that optimization of the liposomes (e.g., addition of dopants) may provide a more dramatic fluorescent response and provide additional consistency and advantages.

V. Detection of Analytes

In some embodiments, the biopolymeric materials of the presently invention find use for the detection of a large variety of analytes including, but not limited to, small molecules, microorganisms, membrane receptors, membrane fragments, volatile organic compounds (VOCs), enzymes, drugs, antibodies, and other relevant materials by the observation of color changes that occur upon analyte binding. The presently claimed invention works under very mild testing conditions, providing the ability to detect small biomolecules in a near natural state and avoiding the risks associated with modification or degradation of the analyte.

In preferred embodiments of the presently claimed invention, a fluorescent response is observed by an optical mechanical device (e.g., high resolution CCD visible light microscopy, total internal reflection microscopy (TIR), and the like).

Additionally, the presently claimed invention can be, if desired, attached to a transducer device. The association of monomer (in some cases self-assembling monomers) materials with transducers has been described using optical fibers (See e.g., Beswick and Pitt, J. Colloid Interface Sci. 124: 146 [1988]; and Zhao and Reichert, Langmuir 8:2785 [1992]), quartz oscillators (See e.g., Furuki and Pu, Thin Solid Films 210:471 [1992]; and Kepley et al., Anal. Chem. 64:3191 [1992]), and electrode surfaces (See e.g., Miyasaka et al., Chem. Lett., p. 627 [1990]; and Bilewicz and Majda, Langmuir 7:2794 [1991]).

In some embodiments, sensitivity can be enhanced by coupling the lipid-polymer to a photoelectric device, fluorometer, or fiber optic tip that can read at two or more specific wavelengths. Additionally, in some embodiments, the detecting device is linked to an alternative signalling device such as a sounding alarm or vibration to provide simple interpretation of the signal.

VI. Immobilization of Biopolymeric Materials

The biopolymeric material of the present invention can be immobilized on a variety of solid supports, including, but not limited to polystyrene, polyethylene, teflon, silica gel beads, hydrophobized silica, mica, filter paper (e.g., nylon, cellulose, and nitrocellulose), glass beads and slides, gold and all separation media such as silica gel, sephadex, and other chromatographic media. In some embodiments, the biopolymeric materials are immobilized in silica glass using the sol-gel process.

Immobilization of the fluorometirc/colorimetric biopolymeric materials of the present invention may be desired to improve their stability, robustness, shelf-life, fluorescent response, color, ease of use, assembly into devices (e.g., arrays), and other desired properties. In some embodiments, placement of fluorescent materials onto a variety of substrates surfaces can be undertaken to create a test method similar to the well-known and easy to use litmus paper test. For example, the reflective properties of nylon filter paper greatly enhance the fluorescent properties of the immobilized polydiacetylene liposomes. Filter paper also increases the stability of the liposomes due to the mesh size. In another example, the liposome embodiment of the present invention has been loaded into the ink cartridge of a ink jet printer and used to print biopolymeric liposome material onto paper as though it were ink. The liposome material present on the paper maintains its fluorescent properties upon binding analytes. This embodiment demonstrates the ease with which patterned arrays can be generated into any desired shape and size.

A. Entrapment of Biopolymeric Material by the Sol-Gel Method

The sol-gel process has been used for entrapping organic molecules such as dyes and biomolecules in silica gels (See e.g., Avnir, Accounts Chem. Res. 28: 328 [1995]). Embodiments of the present invention provide immobilization of spherical, bilayer lipid aggregates, and liposomes using an aqueous sol-gel procedure. These molecular structures, and particularly liposomes, composed of biological or biomimetic (i.e., mimics nature) lipids, are fairly robust under aqueous conditions and ambient temperatures, but can easily degrade in the presence of organic solvents and high temperatures. The sol-gel process provides a facile method of immobilizing molecular aggregates with no detectable structure modification, creating robust structures that are easily fabricated into any desired size or shape.

Silica sol-gel material is prepared by sonicating tetramethylorthosilicate, water, and hydrochloric acid under chilled conditions until a single phased solution is obtained. The use of metal oxides, other than tetramethylorthosilicate, are contemplated by the present invention, so long as they facilitate the entrapment and form substantially transparent glass material. Such metal oxides include, but are not limited to, silicates, titanates, aluminates, ormosils, and others. Buffer is added to the acidic solution under cooling conditions. The biopolymeric materials, generated as described above, are mixed into the buffered sol solution. This composite is poured into a desired molding structure and allowed to gel at ambient temperatures. The present invention is not limited by the type of molding structure used, as it is contemplated that a variety of structures can be applied to generate gels of any desired size and shape including, but not limited to, cuvettes, flat surfaces for generating thin films, plastic, ceramic, and metal moldings to generate badges, etc. It is not intended that the present invention be limited to gelation at ambient temperatures, as any temperature range that facilitates the production of functional analyte-detecting gels is contemplated.

In one embodiment, liposomes are incorporated into sol-gel glass, although incorporation of any biopolymeric structure is contemplated by the present invention. Following the sol-gel procedure as described above, gelation typically occurs within a few minutes.

In another embodiment, sol-gel materials of various thicknesses will possess unique sensitivities to analytes. It is contemplated that thicker films have a higher surface to volume ratio and therefore may require a higher concentration of analyte to trigger the fluorescent transition. Furthermore, the pore size of the sol-gel material can be adjusted to act as a size selective barrier against undesired materials and analytes. In certain cases, it may be desirable to have a sensor that is less sensitive. This will prevent “false-triggering” in the presence of low levels of analytes where such levels are not relevant (e.g., safe levels of VOCs). Thus, it is contemplated that the sensor will be fine-tuned to only trigger at or above pre-designated levels of the analyte or interest in particular settings.

Furthermore, the gelling conditions of the sol-gel preparation can be optimized by varying gelling temperatures, gel materials, and drying conditions to generate material with desired pore sizes. Varying the crosslinker density of the material also provides control over pore size. Pore sizes from nanometers to hundreds of nanometers or greater are contemplated by the present invention. Some gels allow size-selective screening of undesired material while maintaining analyte access to the ligand. Also, the sol-gel technique allows structures to be formed that can be molded into any desirable shape, including, but not limited to, cartridges, coatings, monoliths, powders, and fibers.

B. Immobilization by Chemical Linkage

In some embodiments of the present invention, the biopolymeric material can be attached to membranes of poly(ether urethanes) or polyacrylonitrile. These membranes are porous, hydrophilic and can be used for affinity separations or immunodiagnosis. The liposomes of the present invention can be coupled to these membranes by first attaching an activating group such as imidizolyl-carbonyl, succinimido, FMP or isocyanate to the membrane which adds rapidly to nucleophiles (e.g., —NH₂, —SH, or —OH groups) present in the liposomes. Thus, any liposome preparation which contains these functionalities can be directly attached to the membrane. This procedure is analogous to the coupling of proteins to membranes, the latter of which is well known in the art (See e.g., Bamford et al., Chromatography 606: 19 [1992]).

A variety of other immobilization techniques known in the art find use with the biopolymeric material of the present invention. For example, materials which have an —SH functionality can also be immobilized directly to gold surfaces, particles, or electrodes via the thiol-gold bond. In this case, a solution of the liposomes containing the —SH group are incubated with the clean gold surface in water for 12-24 hours with stirring at room temperature. Also, materials can be immobilized to silicon chips or silica gel (e.g., silicon dioxide). Furthermore, materials containing —NH₂ functionalities can also be immobilized onto surfaces with standard glutaraldehyde coupling reactions that are often used with the immobilization of proteins.

VII. Arrays

Certain preferred embodiments of the present invention contemplate the generation of arrays of the biopolymeric assay materials with different headgroup chemistries, ligands, dopants, monomers or other properties within a single device (e.g., microchip, microwell, glass slide, etc.) to increase selectivity, sensitivity, quantitation, ease of use, among other desired characteristics and qualities. By using the array format, several advantages can be realized that overcome the shortcomings of a single sensor approach. These include the ability to use partially selective sensors and to measure multicomponent samples. This offers the possibility of sensing a specific analyte in the presence of an interfering background, or to monitor two or more analytes of interest at the same time. In some embodiments, the sensitivities of a given lipid to a given solvent can be determined in order to generate identifiable fingerprints characteristic of each solvent. The higher the number of elements in the array, the greater the chance of a positive identification for a given analyte. By immobilizing the biopolymeric material, materials of any desired size and shape can be created and incorporated into a small, easily read and interpretable device.

In preferred embodiments, a plurality of biopolymeric materials specific for one or more particular analytes are arrayed on a solid surface in polar coordinates such that a grid of biopolymeric materials specific for one or more particular analytes is created. In certain of these preferred embodiments, arrays of specific domains are produced that allow sampling of multiple analytes in a single sample. For example, a 100×100 μm array can be created to produce a biopolymeric assay with a minimum dynamic range of from 14 bits (0-16,000) to 17 bits (0-128,000). Thus, the present invention further contemplates that a 10×10 array of these 10,000 sq. μm detectors being manufactured by molecular film deposition techniques could be used to evaluate 100 different pathogens or chemical species in a single experiment. The present invention is obviously not limited by the size of the biopolymeric assay surfaces employed. By way of further example, a 2.5×7.5 cm glass slide could be used to create an array of 10,000 domains of 100×100 μm arrays for pathogen or high throughput screening applications. A 100 μm×100 μm array will contain from 16,000 to 128,000 fluorescent reporter polymer units. Each activated domain produces greater than 20,000 cps. A scanning device, including optical detectors, could readout 100 such detection domains in about 3 sec. Incorporation of fiber optic design elements into the biopolymeric assay detector systems could reduce scanning time to about to 30 msec per 100 detection domains.

VII. Total Internal Reflection Microscopy

Preferred embodiments of the present invention use total internal reflection microscopy (TIRM) techniques to provide optical techniques for monitoring the instantaneous separation distance between a microscopic object (e.g., a liposome) and a flat plate. Changes in distance as small as 1 nm can be detected. To determine the instantaneous separation distance, the intensity of light scattered by the sphere when it's illuminated by an evanescent wave is measured. When a microscopic object with a refractive index different from that of the water settles near an interface at which total internal reflection occurs, some of the evanescent wave is scattered this situation is called “frustrated” total internal reflection. Because of the nonuniform illumination of the water by the evanescent wave, the amount of light scattered by the sphere is exquisitely sensitive to its proximity to the interface.

A number of comprehensive review articles concerning total internal reflection fluorescence microscopy (TIRM) have been published. (See e.g., Evanescent-wave microscopy: A new tool to gain insight into the control of transmitter release., M. Oheim et al., Phil. Transactions Royal Soc. London, Series B: 354:307-318 [1999]; D. Axelrod, Light Microscopy in Biology, Lacey, A. (ed), Oxford University Press, New York, pp. 399-423 [1999]; M. Born and E. Wolf, Principles of Optics, 7th Edition, Cambridge University Press, Cambridge, United Kingdom, pp. 49-53 [1999]; E. Hecht, Optics, Addison Wesley Longman, Incorporated, New York, 121-127 [1998]; L. Tamm, Optical Microscopy: Emerging Methods and Applications, Herman, B. and Lemasters, J. (eds), Academic Press, New York, pp. 295-337 [1993]; and D. Axelrod et al., Topics in Fluorescence Spectroscopy, Volume 3: Biochemical Applications, Lakowicz, J. (ed), Plenum Press, New York, 289-343 [1992]; Evanescent-wave microscopy: a simple optical configuration., J. Murray and D. Eshel, J. Microscopy, 167:49-62 [1992]).

Experimental

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); ° C. (degrees Centigrade); μCi (microcurie); mN (millinewton); Å (angstrom); kDa (kilodalton); ppm (parts per million); N (newton); wt % (percent by weight); aq. (aqueous); J (Joule); UV (ultraviolet); XPS (x-ray photoelectron spectroscopy); PDA (diacetylene monomer); PCA (pentacosadiynoic acid monomer); DCDA (docosadynoic acid); SA-PDA (sialic acid-derived PDA); OTS (octadecyltrichlorosilane); VOC (volatile organic chemical); pH (hydrogen ion concentration); EDC (ethylcarboiimide hydrochloride); AFM (atomic force microscope); Hz (Hertz); LB (Langmuir-Blodgett); NHS (N-hydroxy succinimide); CO₂ (carbon dioxide); MgSO₄ (magnesium sulfate); CdCl₂ (cadmium chloride); MeOH (methanol); Be (beryllium ions); Mg (magnesium ions); Ca (calcium ions); Ba (barium ions); N₂ (nitrogen gas); Sigma (Sigma Chemical Co., St. Louis, Mo.); Perkin-Elmer (Perkin-Elmer Co., Norwalk, Conn.); Fisher (Fisher Scientific, Pittsburgh, Pa.); and Farchan Laboratories (Farchan Laboratories, Inc., Gainesville, Fla.); Park Scientific Instrument (Park Scientific Instruments, Sunnyvale, Calif.); Biorad (BioRad Laboratories, Hercules, Calif.); Gelman (Gelman Sciences, Ann Arbor, Mich.); Pierce (Pierce, Rockford, III); and Bellco Glass (Bellco Glass Inc., Vineland, N.J.).

### EXAMPLE 1

### Biopolymeric Material Preparation

I. Production of Liposomes

The monomers (in some cases self-assembling monomers) to be incorporated into the liposomes are dissolved in solvent (e.g., chloroform for diacetylenes and methanol for ganglioside G_{M1}). Many other volatile solvents find use in the present invention, including, but not limited to, benzene, hexane, and ethylacetate. The solvent solutions are mixed in appropriate volumes in a brown vial (i.e., to prevent light interference during the upcoming drying steps) to achieve the desired lipid mixture (e.g., 5% by mole of G_{M1}, 95% diacetylenes) and a total lipid content of approximately 2 μmol. The solvent is then evaporated by rotary evaporation or with a stream of nitrogen gas. The dried lipids are resuspended in sufficient de-ionized water to produce a 1-15 mM solution of lipid. The solution is then sonicated for 15-60 minutes with a probe sonicator (Fisher sonic dismembrator model 300, 50% output, microtip) as described by New (New, supra). The solution is heated during the sonication (in most cases the sonicating process alone provides sufficient heat) to a temperature above the phase transition of the lipids used (typically 30-90° C.). The resulting mixture is filtered through a 0.8 micromole nylon filter (Gelman) or through a 5 mm Millipore Millex-SV filter and cooled to 4° C. for storage or is polymerized. In one embodiment, prior to polymerization, oxygen in the solution is removed by bubbling nitrogen through the sample for 5-10 minutes.

Polymerization of the stirred liposome solution is conducted in a 1 cm quartz cuvette with a small 254 nm UV-lamp (pen-ray, energy: 1600 microwatt/cm²) at a distance of 3 cm. The chamber is purged with nitrogen during the polymerization to replace all oxygen and to cool the sample. Polymerization times are varied between 5 and 30 minutes depending on the desired properties (e.g., degree of polymerization) of the liposomes. In other embodiments, the solution is placed in a UV-chamber, without purging, and exposed to 0.3-20 J/cm² of ultraviolet radiation, preferably 1.6 J/cm², for 5-30 minutes.

In some embodiments, polymerization is conducted in a multi-chambered plate (e.g., ELISA plate). Approximately 200 μl of sonicated liposome solution are placed in each well of the plate. The plate is placed under a UV lamp with the distance between the plate and the lamp kept at 3 cm. Irradiation times typically last for a minute.

II. Production of Films

Polydiacetylene films are formed in a standard Langmuir-Blodgett trough (See e.g., Roberts, Langmuir Blodgett Films, Plenum, N.Y. [1990]). The trough is filled with water to create a surface for the film. Distilled water is purified with a millipore water purifier with the resistivity of 18.2 M-Ohm. In some embodiments, diacetylene monomers (e.g., 5,7-docosadiynoic acid, 10,12-pentacosadiynoic acid [Farchan Laboratories], 5,7-pentacosadiynoic acid, combinations thereof, or other self assembling monomers), dissolved in a solvent spreading agent (e.g., spectral grade chloroform [Fisher]), are layered onto the aqueous surface with a syringe, to form a continuous film. Monomers prepared in the concentration range of 1.0 to 2.5 mM, are kept at a temperature of 4° C. in the dark, and are allowed to equilibrate at room temperature before being used in experiments.

Once layered on the water surface, the film is physically compressed using moveable barriers to form a tightly-packed monolayer of the monomers. The monolayer is compressed to its tightest packed form (i.e., until a film surface pressure of 20-40 mN/m is achieved). Following compression, the film is polymerized. Certain embodiments (e.g., embodiments with dopants) of the present invention may require surface pressure compression greater or less than 20-40 mN/m.

Ultraviolet irradiation is used to polymerize the monomers, although other means of polymerization are available (e.g., gamma irradiation, x-ray irradiation, and electron beam exposure). Pressure is maintained on the film with the moveable barriers throughout the irradiation process at surface pressure of 20-40 mN/m. An ultraviolet lamp is placed 20 cm or farther from the film and trough. If the lamp is placed closer to the film, damage to the diacetylene film may occur due to the effects of heating the film. The film is exposed to ultraviolet light with a wavelength of approximately 254 nm for approximately one minute. The polymerization is confirmed by observing the blue color acquired upon polymerized diacetylene formation and detecting the linear striations typical of polymerized diacetylene films with a polarizing optical microscope.

III. Production of Tubules

Self-assembling monomers to be incorporated into the tubules are dissolved in solvent, mixed together, evaporated, and resuspended in water as described above for liposomes. One to ten percent by volume of ethanol is added to the solution, although other organic solvents are contemplated by the present invention. The solution is then sonicated (with heating if necessary), filtered, cooled, and polymerized as described above for liposomes.

### EXAMPLE 2

### Examination of Biopolymeric Materials

I. Optical Microscopy and X-Ray Spectroscopy

Diacetylene films are prepared in a Langmuir Blodgett trough as described above using a combination of PDA monomers and sialic acid-derived PDA monomers. The floating polymerized assembly are lifted by the horizontal touch method onto a glass slide previously coated with a monolayer of octadecyltrichlorosilane (OTS) as described (Maoz and Sagiv, J. Colloid Interface Sci. 100:465 [1984]).

The slide is then examined by optical microscopy with the use of crossed polarizers as described (Day and Lando, Macromolecules 13: 1478 [1980]). In preferred embodiments, films exhibit a high degree of order over a macroscopic range (i.e., 50 to 150 μM) and large domains up to 150 μM will be visible (1 cm=10 μM).

In preferred embodiments, the films are further characterized by angle-resolved x-ray photoelectron spectroscopy (XPS) and ellipsometry. The XPS results should indicate that the amide nitrogen atoms and the carbonyl carbon atoms of the head groups are localized at the surface relative to the methylene carbons of the lipid chains, thus demonstrating that the sialoside head group is presented at the surface of the film. Ellipsometric analysis of the polydiacetylene monolayer coated on HF-treated silicon should also indicate a film thickness of approximately 40 Å, in agreement with the expected value based on molecular modeling.

II. Atomic Force Microscopy

In some embodiments, in situ atomic force microscopy is used to reveal the morphology, surface topography, and growth and dissolution characteristics of microscopic biopolymeric crystals, and allows dynamic observations of nucleation events and the determination. Studies are conducted using standard techniques for in situ studies as described by Binnig et al. (Binnig et al., Phys. Rev. Lett., 12:930 [1986]; and Binnig et al., Europhys. Lett. 3:1281 [1987])

In preferred embodiments, two different atomic force microscopes are used to study biopolymeric material production. The present invention contemplates that images larger than 1 μm² be acquired with a commercially available instrument (e.g., Si ultralevers, Park Scientific Instrument). Commercially available photolithographically patterned glass slides (Bellco Glass) are used to allow imaging of the exact same region of the film after each temperature step. Images smaller than 1 μm² are taken with a home-built AFM (Kolbe et al., Ultramicroscopy 42-44: 1113 [1992]). Si₃N₄ cantilevers with a nominal force constant of 0.1 N/m are used (Park Scientific Instruments). Both microscopes are operated in contact mode, and in the latter case a four-quadrant position-sensitive photodiode allows the measurement of the cantilever bending and twisting simultaneously. All images are acquired in contact mode under ambient conditions.

### EXAMPLE 3

### Optimization of Biopolymeric Materials

The present invention provides a variety of different biopolymeric material forms (e.g., liposomes, films, tubules, etc.), with and without dopant materials, with a variety of ligands, and immobilized in a variety of forms. For each of these embodiments, it is possible to optimize the biopolymeric material to maximize analyte sensitivity, robustness, fluorometric response, and other desired factors. Described below are a few illustrative examples of such optimization. These examples are intended to merely illustrate the flexibility of the present invention. It is not intended that the present invention be limited to these particular embodiments.

I. Mixed Monomers

The biopolymeric material of the presently claimed invention can comprise a sample of pure monomers (e.g., pure PDA) or can comprise mixed monomers (e.g., PDA with ganglioside G_{M1} or dopant). Optimization of the percent composition of mixed monomers can be undertaken to provide analyte-detecting biopolymeric materials with desired properties. An example of such optimization is provided below for the detection of cholera toxin with a ganglioside ligand.

To evaluate the fluorescent response of G_{M1}/PDA films, different concentration combinations of ligand (i.e., G_{M1}) and PDA are tested. If too much ligand molecule is added (i.e., low concentration of polymerized lipid), the films are unstable and have high background levels. If the films have too much polymerized lipid molecule, they are too stable and the fluorescence does not occur well. In search of the G_{M1}/PDA biosensor composition capable of displaying maximal response, a series of PDA monolayer films are transferred to OTS coated glass slides. The films are evaluated by exposure to cholera toxin and the fluorescent response is measured using UV-Vis spectroscopy. Simple comparisons can be made to determine the optimal proportions of biopolymeric materials for peak fluorescent response.

II. Optimization of Subionic Phase

The ionic content of the aqueous subphase has significant impact on the properties of Langmuir monolayers. The presence of cationic species strengthens the electrostatic interaction of monolayer with anionic headgroups and consequently stabilized the film (Gaines, Insoluble Monolayers at Liquid-Gas Interface, Interscience Publishers, New York, pp 291-299 [1966]). For example, many divalent ions (Be, Mg, Ca, Ba, and Cd) have been shown to have an impact on the isotherms of PDA monomers through salt formation, which influences the packing of molecules on a basis of ion size and charge. From the above discussion, optimization of the subionic phase for particular biopolymeric assay materials can be resolved.

III. Optimization of Subphase pH

For acidic PDA molecules, an increase in pH is contemplated to result in the ionization of PDA molecules and the introduction of substantial charge along the monolayer interface. At high pH (pH 9.2), the films became very expanded as a result of electrostatic repulsion between the adjacent PDA molecules. Compression of such films to form monolayers is difficult. The present invention contemplates that the addition of compounds such as G_{M1} (i.e., which is acidic) into the PDA mixture at high pH will thus be unfavorable. From the above discussion, optimization of the subionic phase for particular biopolymeric assay materials can be resolved.

IV. Optimization of Subphase Temperature

During film production, an increase in temperature usually results in higher surface pressure, an enlargement of the expanded region, and a shift in the phase transition point towards the low molecular area direction in π/A isotherms (Birdi, Lipid and Biopolymer Monolayers at Liquid Interfaces, Plenum Press, New York [1989]). This effect stems from the higher flexibility of hydrocarbon tails of lipids at high temperature as a result of thermal agitation, and can be analyzed with the two-dimensional Clausius-Clapeyron equation (Birdi, supra). Monolayer films containing PDA, however, typically experience film collapse during compression. Consequently, the evaluation of the subphase temperature effect has to take this phenomenon into consideration. A decrease in subphase temperature usually results in an increase in surface pressure and the isotherm shape is changed. Isotherms at low temperature typically exhibit increasing liquid-solid phase transition features.

V. Position of the Monomer Polymerizable Group

A comparison of the fluorescent responses of 10,12-pentacosadiynoic acid liposomes and 5,7-docosadiynoic acid liposomes to analyte can be conducted to determine the effect of the position of the polymerizable group within the monomers. G_{M1} ligands are incorporated into each type of liposome to analyze the detection of cholera toxin. The ganglioside G_{M1} is mixed at 5 mol % with the diacetylene “matrix lipid” monomers. Liposomes are prepared using the probe sonication method and polymerized by UV irradiation (254 nm).

It is contemplated that addition of cholera toxin to the liposomes composing 5% G_{M1} and 95% 5,7-docosadiynoic acid, will cause a strong fluorescent response in the solution. However, the present invention further contemplates that addition of cholera toxin to liposomes comprising 10,12-pentacosadiynoic and ganglioside G_{M1} will cause the fluorescent response to be significantly reduced. Although it is not necessary to understand the mechanism in order to use the present invention, and it is not intended that the present invention be so limited, the present invention contemplates that the predicted sensitivity of the 5,7-docosadiynoic acid liposomes arises from the positioning of the optical reporter group nearer to the interface (i.e., three methylene units compared to eight). It has been shown by Fourier transform IR spectroscopy that small rotations about the C-C bond β to the polymer backbone are sufficient to change the effective conjugated length (Berman et al., Science 259: 515 [1995]).

### EXAMPLE 4

### Incorporation, Optimization, and Properties of Dopants

Each time a new sensor system is designed, the amount of PDA, derivatized PDA, and ligand (e.g., ganglioside) are varied to create the optimal sensor. Although 0-100% amounts are typically used for testing, optimal systems appear to use 5-15% ligands, 0-95% PDA, and 0-95% derivatized PDA. The percent of each component depends on the system, the needed stability, and the needed sensitivity. Certain embodiments of the present invention may incorporate more than one type of dopant into the biopolymeric material.

In one embodiment of the present invention, directed to a thin film assembly for detecting toxins, an organic mixed solvent (chloroform:methanol 2:1) containing 2-5% sialic acid- or lactose-derivatized PDA, 90-93% PDA, and 5% G_{M1} or G_{T1b} is dispersed onto the surface of a Langmuir-Blodgett (LB) trough from RSV (Helsinki, Finland) containing 1×10⁻³ M Cd²⁺ as the subphase. The substances are equilibrated at room temperature for 30-60 minutes to allow organic solvent to evaporate, compressed to the solid-analogous phase, and transferred to the glass slides pre-coated with octadecyltrichlorosilane through vertical dipping at the speed of 5 mm/min. The resulting LB films are polymerized 1 minute per side with a UVP mineral light (Fisher).

I. Incorporation of Dopant into Biopolymeric Material

Amino-acid derivatized diacetylene dopants are incorporated into fluorescent liposomes. The lipids (i.e., the dopants and the diacetylene monomers) are first dissolved in chloroform, and an aliquot is transferred to the reaction vial. The organic solvent is blown out by using N₂ gas, and an appropriate amount of water is added to bring the lipid concentration to approximately 1 mM. Bath sonication is used to break down the white precipitate to form liposomes. Typical sonication times vary from 1 hour to 5 hours, dependent on the type of dopants being used. During sonication, the temperature is carefully raised to approximately 80° C. to facilitate the formation of the liposomes. Sonication should continue until the solutions become clear. The hot solutions are immediately filtered though a 5 μM Millipore Millex-SV filter to remove any impurity that may be present in the solution. The solutions are stored at 4° C. overnight before use.

Polymerization of PDA and PDA derivative liposomes/films is achieved through a hand held UV lamp. For liposomes, the liposomes are placed in ELISA plates with each well containing 200 μL solution. The plate is placed under a UV lamp while the distance between the plate and the lamp is kept at about 3 cm. The irradiation typically lasts for a about minute. The final liposomes contain the amino-acid derivatized diacetylene dopant.

II. Optimization of Dopant Concentration

Films comprising PDA, G_{M1} (i.e., the ligand) and sialic acid-derived PDA (i.e., the dopant) are generated as described in Example 3, Section I for the detection of cholera toxin. For the optimal detection of cholera toxin, both SA-PDA and G_{M1} need be present in the films. The present invention contemplates optimizing the levels of SA-PDA and G_{M1} for peak fluorescent response. Although it is not necessary to understand the mechanism in order to use the present invention, and it is not intended that the present invention be so limited, it is contemplated that the function of SA-PDA is to provide the metastable state of the films for biomolecular recognition through a stress-induced mechanism (See, Charych et al., Chem. and Biol. 3: 113 [1996]).

### EXAMPLE 5

### Attachment of Ligands

Ligands can be covalently linked to the head groups of monomers (e.g., sialic acid linked to diacetylene monomers), or can be covalently linked to the surface of polymerized materials (e.g., proteins and antibodies with multiple amine and thiol linkages to the material surface), or can be non-covalently incorporated into the biopolymeric material (e.g., ganglioside incorporated into the membrane of films and liposomes).

The monomers can be synthesized to contain a large variety of chemical head-group functionalities using synthesis techniques common in the art. In some embodiments, the ligands are then joined to the monomers through chemical reaction with these functionalities using synthesis methods well known in the art. The functionalities include, but are not limited to, esters, ethers, amino, amides, thiols, or combinations thereof. Alternately, many ligands can be incorporated into the matrix without covalent linkage to the surfactants (e.g., membrane proteins and molecules with hydrophobic regions such as gangliosides and lipoproteins).

Specific applications of the presently claimed invention are described below to illustrate the broad range of ligands that can be associated with the inventive biopolymeric material. These examples are intended to merely illustrate the broad applicability of the present invention and are not intended to limit the present invention to these particular embodiments.

I. Sialic Acid

Sialic acid is attached as a ligand to diacetylene monomers. Several synthesis methods well known in the art can be used, many of which have general applicability to the attachment of carbohydrates to the inventive biopolymeric materials.

In one embodiment, PDA (1.0 g, 2.7 mmol in chloroform) is reacted with N-hydroxy succinimide (NHS) (0.345 g, 3.0 mmol) and 1-(3-dimethylaminopropyl)-3-ethylcarbodiimide hydrochloride (EDC) (0.596 g, 3.1 mmol). The solution is stirred for 2 hours followed by evaporation of the chloroform. The residue is extracted with diethyl ether and water. The organic layer is dried with magnesium sulfate (MgSO₄) and filtered. The solvent is then evaporated by rotary evaporation to give 1.21 g of N-succinimidyl-PDA (NHS-PDA). Ethanolamine (0.200 ml, 2.9 mmol) is added to a solution of NHS-PDA (1.21 g in 50 ml of chloroform), followed by triethylamine (0.350 ml, 2.5 mmol) and stirred for two hours at room temperature. The solvent is evaporated and the residue is purified by silica gel chromatography (2:1 EtOAc:hexane, R_{f}=0.15) to give 0.99 g of N-(2-hydroxyethyl)-PDA.

Tetraethylene glycol diamine (1.26 g, 6.60 mmol) in 25 ml of chloroform is added to a solution of N-succinimidyl-PDA (0.603 g, 1.28 mmol) in 20 ml of chloroform, dropwise, with stirring, over a period of 30 minutes. The reaction is stirred for an additional 30 minutes before removal of the solvent by rotary evaporation. The residue is dissolved in EtoAc and then extracted twice with water. The organic layer is dried with MgSO₄, and the solvent is removed by rotary evaporation. The extract is purified by silica gel chromatography (20:1 CHCl₃:MeOH, R_{f}=0.20) to give 3.72 g of N-(11-amino-3,6,9-trioxyundecanyl)-PDA.

Two ml of acetic anhydride is added to a cooled solution of ethyl-5-N-acetyl-2,6-anhydro-3,5-dideoxy-2-C-(2-propenyl)-D-erythro-L-mannonononate (0.47 g, 1.30 mmol) in 1.7 ml of pyridine under nitrogen, with stirring. The reaction is allowed to warm to room temperature overnight. After 18 hours, the solvents are removed under reduced pressure at ambient temperature, to yield a crude viscous oil. The oil is solidified by repeated evaporation from toluene. The crude solid is flash chromatographed over silica with ethylacetate as eluent, to produce 0.58 g of ethyl-5-N-acetyl-4,7,8,9-tetra-O-acetyl-3,5-dideoxy-2-C-(2-propenyl)-D-erythro-L-manno-nononate.

A solution of ethyl-5-N-acetyl-4,7,8,9-tetra-O-acetyl-3,5-dideoxy-2-C-(2-propenyl)-Derythro-L-manno-nononate (0.38 g, 0.72 mmol) in 10 ml of acetone is cooled to −78° C. while protected from moisture with a CaCl₂ drying tube. Ozone is aspirated into the solution until a blue color persists for 5 minutes. The reaction is purged with O₂ to dissipate the excess O₃, followed by warming to 5° C. Excess Jones' reagent (7 drops) is added until a rust orange color persists, then the reaction is warmed to ambient temperature. After several minutes, ethanol is added dropwise to consume the excess oxidant. The green precipitate is filtered and washed with acetone several times. The combined filtrates are evaporated in vacuuo and dissolved in ethylacetate. The solution is extracted with saturated aqueous NaHCO₃ solution three times. The combined aqueous layers are acidified with concentrated HCl and extracted 5 times with methylene chloride. The combined methylene chloride extracts are dried with MgSO₄, filtered and evaporated in vacuuo to give ethyl-5-N-acetyl-4,7,8,9-tetra-O-acetyl-3,5-dideoxy-2-C-(acetic acid)-D-erythro-L-manno-nonate.

Ethyl-5-N-acetyl-4,7,8,9-tetra-O-acetyl-3,5-dideoxy-2-C-(acetic acid)-D-erythro-Lmanno-nonate (0.194 g, 0.35 mmol) is added to a cooled solution (5° C.) NHS (0.058 g, 0.50 mmol) and EDC (0.096 g, 0.50 mmol) in 2 ml of chloroform, under nitrogen. The reaction is warmed to ambient temperature with stirring for 5 hours. The reaction is then diluted with 15 ml of chloroform and washed with 1 N HCl (aq.), twice; saturated (aq.) sodium bicarbonate, twice; and saturated (aq.) sodium chloride, once. The organic layer is dried over MgSO₄, filtered, and evaporated to form ethyl-5-N-acetyl-4,7,8,9-tetra-O-acetyl-3,5-dideoxy-2-C-(Nsuccinimidylacetate)-D-erythro-L-manno-nononate.

Ethyl-5-N-acetyl-4,7,8,9-tetra-O-acetyl-3,5-dideoxy-2-C-(N-succinimidylacetate)-Derythro-L-manno-nononate (0.143 g, 0.22 mmol) and N-(1-amino-3,6,9-trioxyundecanyl)PDA (0.133 g, 0.24 mmol) are dissolved in 2 ml of chloroform and the reaction is sealed and stirred for 56 hours. The solution is diluted with 15 ml of chloroform and washed with sodium chloride saturated 1N HCl (aq.), twice; saturated (aq.) sodium bicarbonate, twice; and saturated (aq.) sodium chloride, once. The organic layer is dried over MgSO₄, filtered, and evaporated to a crude semi-solid. The material is flash chromatographed over silica (20:1 CHCl₃:MeOH), producing ethyl-5-N-acetyl-4,5,8,9-tetra-O-acetyl-3,5-dideoxy-2-C-[(N-11′-(PDA)-3′,6′,9′-trioxyundecanyl) acedamido]-D-erythro-L-manno-nononate.

The sialic acid derived-PDA is formed by dissolving ethyl-5-N-acetyl-4,5,8,9-tetra-Oacetyl-3,5-dideoxy-2-C-[(N-11′-(PDA)-3′,6′,9′-trioxyundecanyl) acedamido]-D-erythro-Lmanno-nononate (0.20 g, 0.19 mmol) in a solution of 4 ml of water and 0.5 ml of methanol containing 0.1 g dissolved sodium hydroxide. The solution is stirred for 3 hours, and ion exchange resin (Biorad AG 50W-X4 H+ form) is added until the solution is acidic to pH paper. The solution is filtered and the filtrate evaporated in vacuo, to produce sialic acid derived-PDA.

II. Carbohydrates

In other embodiments, carbohydrates (i.e., including sialic acid) can be modified by a three-step procedure to produce N-allyl glycosides. The N-allyl glycosides can then be easily linked to other molecules (e.g., PDA) using simple chemical synthesis methods known in the art. This method provides a means to incorporate a broad range of carbohydrates into biopolymeric material (and thus provides a means to detect a broad range of analytes). First, oligosaccharides are dissolved in neat allyl amine (water may be added if necessary and does not adversely affect the yield) producing a 0.5-0.1 M solution. The reaction is stopped and stirred for at least 48 hours. Upon complete conversion of the starting material into amino glycoside product, the solvent is removed by evaporation and the crude solid is treated with toluene and evaporated to dryness several times. The solid is then chilled in an ice bath and a solution of 60% pyridine, 40% acetic anhydride is added to give a solution containing five hundred mole percent excess of acetic anhydride. The reaction is protected from moisture, stirred and allowed to warm to ambient temperature overnight. The solvents are removed by evaporation and the residue is dissolved in toluene and dried by evaporation several times. The crude product is purified by flash chromatography producing the peracetylated NAc-allyl glycoside form of the free sugars.

The peracetylated NAc-allyl glycosides are then dissolved in anhydrous methanol to give a 0.1-0.01 M solution. Several drops of 1 N NaOMe in MeOH are added and the reaction stirred at ambient temperature for 3 hours. Enough Dowex 50 resin (H+ form) is added to neutralize the base, then the solution is filtered and evaporated to dryness (purification by recrystallization can be conducted if desired). The products are the N-allyl glycoslamide form of the carbohydrates. These synthesis reactions have produced the N-allyl glycoslamide forms of a variety of carbohydrates, including, but not limited to, glucose, NAc-glucosamine, fucose, lactose, tri-NAc-Chitotriose, Sulfo Lewis^{X} analog, and Sialyl Lewis^{X} analog. Skilled artisans will appreciate the general applicability of this method to the attachment of a broad range of carbohydrates to diacetylene lipids.

III. Ganglioside G_{M1}

Ganglioside G_{M1} presents an example of non-synthetic incorporation of a ligand without covalent attachment to the monomers (in some cases self-assembling monomers). Ganglioside G_{M1} is introduced in the biopolymeric material by combining a solution of methanol dissolved ganglioside G_{M1} (Sigma) with chloroform dissolved PDA, and dried. The ganglioside contains a hydrophobic region that facilitates its incorporation into self-assembling surfactant structures. Thus, when the dried solutions are resuspended in deionized water, the resulting structures contain a mixture of ganglioside and PDA. Liposomes and other forms are produced from the resuspended mixture as described in Example 1. Although the ganglioside does not contain a polymerizable group, the ganglioside becomes embedded in the polymerized matrix created by the cross-linking of the diacetylenes. Similar methods can be used for the incorporation of other ligands that contain hydrophobic regions (e.g., transmembrane proteins and lipoproteins).

IV. Proteins

The NHS-PDA, as generated above, thiol-linked PDA, and other methods known in the art provide functional groups for the attachment of proteins and antibodies. The NHS or thiol-linked monomers are incorporated into the desired aggregate and polymerized. The NHS or thiol functional groups then provide a surface reaction site for covalent linkage to proteins and antibodies using chemical synthesis reactions standard in the art. In another embodiment, a hydrazide functional group can be placed on PDA, allowing linkage to aldehydes and ketone groups of proteins and antibodies. These embodiments provide a means to incorporate an extremely broad array of proteins and antibodies onto the biopolymeric material. Specific examples are provided below. These examples are intended to merely illustrate the broad applicability of the present invention and are not intended to limit the present invention to these particular embodiments.

A. Hexokinase

NHS-PDA lipid is synthesized as described above. In brief, 1.00 g 10,12-pentacosadiynoic acid (Farchan, Gainesville, Fla.) is dissolved in CHCl₃, to which 0.345 g N-hydroxysuccinimide (NHS) and 0.596 g 1-(3-dimethylaminopropyl)-3-ethylcarbodiimide hydrochloride are added. The solution is stirred at room temperature for two hours, followed by removal of CHCl₃ using a rotavap. The residue is extracted with EtOAC and water. After separation, the organic layer is dried with MgSO₄ and filtered, followed by solvent removal. The raw product is then recrystallized twice with CHCl₃, and confirmed by FT-IR.

The 1:1 (molar ratio) PDA/NHS-PDA chloroform solution is spread on the aqueous subphase on a Langmuir-Blodgett trough (KSV mini-trough, KSV Instruments, Inc., Finland) by using a microsyringe (subphase temperature is maintained at 5° C.). The organic solvent is allowed to evaporate by resting the solution for 20 min. The films are compressed to compact monolayer level and then transferred by vertical deposition to glass slides coated with octadecyltrichlorosilane (OTS). The compression and dipping speed is maintained at 5 mm/min. Three layers are deposited onto the glass slide to provide enough fluorometric/colorimetric signal for detection after polymerization and to ensure the hydrophilic surface is exposed to solution.

The preparation of stable PDA monolayer films before enzyme immobilization is critical for low background and enhanced reproducibility of the sensors. The Langmuir monolayer trough provide a method to measure film stability through the evaluation of the surface collapse pressure of the monolayers. Mixed films (i.e., films with PDA and NHS-PDA) are contemplated to be more stable than the monolayers consisting of one component and thus more suitable for enzyme immobilization. Although it is not necessary to understand the mechanism in order to use the present invention, and it is not intended that the present invention be so limited, it is contemplated that the interactions are more favorable in mixed monolayers, presumably due to the optimal spatial arrangements that allow head groups of different size to pack closely. Besides mechanical stability, the monolayers should possess desirable optical properties to be suitable as sensors.

In one embodiment, a yeast hexokinase suspension (E.C. 2.7.1.1, from Boehringer Mannheim GmbH, Germany) is spun in a microcentrifuge to remove saturated ammonium sulfate. The protein is resolubilized in 0.1 M phosphate buffer (pH 8.0) to give approximately 1 mg/ml concentration, and dialyzed against the same buffer using a Slide-A-Lyzer dialysis cassette (Pierce) for 3 hours. The PDA monolayer slides are cut into 0.7 cm×2.5 cm rectangular pieces, and incubated in the hexokinase solution at 4° C. for 1 hr. The monolayer chips are then rinsed with deionized water and immersed into 0.1 M ethanolamine for 10 min to terminate the reaction. The chips are rinsed again with deionized water and air dried. Polymerization is conducted by irradiating the films with a hand held UV lamp. The irradiation time is 6 minutes each side. Extended irradiation times are not desirable.

B. Antibodies

Commercially obtained diacetylene are filtered to remove the insoluble impurities (e.g., polymerized form) and converted chemically to NHS-PDA as described above. Appropriate amounts of NHS-PDA and other forms of PDA derivatives (e.g., dopants or ligands) are mixed to give the desired molar ratio. The solution is dried using N₂ gas, so a thin layer of white material deposited on the bottom of the vial. Deionized water is added to bring the total concentration of lipid to approximately 1 mM. The solution is sonicated by using either a probe sonicator for approximately 20 minutes or a bath sonicator for over 2 hours until a clear solution is obtained. The solution is filtered through 5 μm filter while hot, then stored at 4° C. overnight.

Prior to cross linking, 0.1 M phosphate buffer (pH 8.5) is added to the liposome solution. Antibody dissolved in a similar buffer are then added, and the solution is stored at 4° C. overnight. Excess antibody is removed by either centrifugation or dialysis. When centrifugation is used, the pellet is resonicated gently using an ice bath. Following association of the antibody to the sonicated material, polymerization is conducted as described for liposomes in Example 1.

V. Others (Amino Acids, Nucleotides, Etc.)

As described above, the attachment of amino acids though amine linkage to diacetylenes can be accomplished. A variety of other means of attaching amino acids to lipids are also known in the art.

The present invention contemplates derivation of the biopolymeric assay materials (e.g., PDA) with a broad range of chemical head groups such as hydrophilic uncharged hydroxyl groups, primary amine functionalities, amino acid derivatives, and hydrophobic groups. These and other modifications are generated by synthesis methods known in the art.

In some embodiments, various other surfactant-linked ligands can be prepared using condensation reactions involving an activated carboxylic acid group and a nucleophilic amino or hydroxy. PDA can be activated with trimethylacetylchloride under anhydrous conditions to form an active asymmetric anhydride. The anhydride can be treated with excess ethylene diamine or ethanolamine to form ethylenediamino-PDA (EDA-PDA) or ethanolamine-PDA (EA-PDA), respectively. One and a half mole equivalents of triethylamine are added as a catalytic base and reactions are allowed to proceed for three hours at room temperature. EDA-PDA and EA-PDA are chromatographically purified using a silica gel column and a chloroform/methanol gradient. The EDA-PDA or EA-PDA are then condensed with free carboxylic acid containing ligands (chemically activated as above) to form the ligand-linked polymerizable surfactants. Representative examples of ligands that can be prepared by this method include, but are not limited to, carbohydrates, nucleotides, and biotin.

The art contains numerous other examples of successful linkage or association of molecules to lipids and membranes. The monomers (in some cases self-assembling monomers) associated with ligands can be of modified chain length or may consist of double or multiple chains. These various combinations of ligands and monomers provide an extremely broad array of biopolymeric materials appropriate for the detection of a broad range of analytes, with the desired fluorescent response, selectivity, and sensitivity.

### EXAMPLE 6

### Fluorometric Analysis

I. Fluorescence Detection

In preferred embodiments the fluorescent response in the biopolymeric assay materials results from contact one or more ligands is detected using photodetector. Fluorescence occurs when light is absorbed from an external (excitation) source by a fluorescent molecule (a fluorophore) and subsequently emitted. The emitted light is of a lower energy (longer wavelength) than the absorbed light because some of the excitation energy is dissipated upon absorption. The characteristic spectral shift between excitation and emission wavelengths of a particular fluorophore is called the Stokes shift. Discrimination between excitation wavelengths and emission wavelengths improves the signal to noise ratio and dynamic range of the detector system by substantially removing background fluorescence and scattered excitation light from fluorophore-specific emission.

### EXAMPLE 7

### Detection of Analytes

The broad range of biopolymeric materials taught by the present invention allow for the detection of numerous analytes. Such analytes range from complex biological organisms (e.g., viruses, bacteria, and parasites) to simple, small organic molecules (e.g., alcohols and sugars). Specific applications of the presently claimed invention are described below to illustrate the broad applicability of the invention to a range of analyte detection systems and to demonstrate its specificity, and ease of use. These examples are intended to merely illustrate the broad applicability of the present invention. It is not intended that the present invention be limited to these particular embodiments.

I. Detection of Influenza Virus

The presently claimed invention provides a superior means of detecting influenza virus compared to currently available technologies. Immunological assays are limited because of the antigenic shift and drift exhibited by the virus. The presently claimed invention detects all varieties of influenza and thus a determination of a patient's exposure to influenza will be definitive, and not limited to a particular strain. Indeed, even newly evolved, uncharacterized influenza strains can be detected.

Sialic acid-linked biopolymeric material is generated as described in Examples 1 and 5. The materials are exposed to influenza virus and fluorometric/colorimetric response is observed as described in Example 6. For liposomes, a 1-10% mixture of sialic acid-linked PCA is incorporated, as previous studies indicated that optimum viral binding occurs for mixtures of 1-10% in liposomes (Spevak et al., J. Am. Chem. Soc. 161: 1146 [1993]).

Competitive inhibition experiments can be conducted to demonstrate the specificity of the ligand-analyte interaction. Experiments are performed as described above, but with a slight excess of α-O-methyl-neuramatic acid, a known inhibitor for influenza virus hemagglutination. The presence of the inhibitor resulted is contemplated to not result in a detectable fluorescent response in the system.

In one embodiment of the present invention, 200 μL of phosphate buffered saline (PBS, pH 7.4) is mixed with 30 μL of the liposome solution (5% sialic acid-derivatized PDA, 9% PDA) in the wells of an ELISA plate. The reaction is started by adding the appropriate amount of influenza virus (in most cases about 30 μL), PBS as a reference, or BSA in PBS (1 mg/mL). Fluorescence is recorded after various times until the color of the liposomes/virus solution is unchanged. In particularly preferred embodiments, the fluorescent response of the assay material is practically instantaneous.

In another embodiment of the present invention, 20 μL of virus solution (54 HAU), 100 μL of PBS buffer and 50 μL of inhibitors in PBS (100 mM α-o-methyl neuramanic acid) are mixed in a microtiter well, and preincubated for 1 hour at room temperature. In two other wells, 150 μL of PBS buffer is mixed with either 20 μL of active virus or with 20 μL of buffer. To start the reaction, 20 μL of the liposome solution (10% sialic acid-PDA) is added to each well. Again, the fluorescence emitted from the assay is monitored over time.

It is contemplated that the influenza virus detection system include additional ligands that recognize and differentiate influenza strains or serotypes from one another and from other pathogens.

The sialic-acid containing biopolymeric materials of the present invention provide means of detecting many other pathogens. In addition to influenza virus, sialic acid has the capability of detecting other analytes including, but not limited to, HIV, chlamydia, reovirus, Streptococcus suis, Salmonella, Sendai virus, mumps, newcastle, myxovirus, and Neisseria meningitidis.

II. Detection of Cholera Toxin

Cholera toxin is an endotoxin of the Gram-negative bacterium Vibrio cholerae that causes potentially lethal diarrheal disease in man. Cholera toxin is composed of two subunits: A (27 kDa) and B (11.6 kDa) with the stoichiometry AB₅. The B components bind specifically to G_{M1} gangliosides on cell surfaces, ultimately leading to translocation of the A₁ fragment through the membrane. Cholera toxin can be recognized by G_{M1}-containing supported lipid membranes and polymerized Langmuir-Blodgett films containing G_{M1} and a carbohydrate “promoter” lipid (i.e., sialic acid-derived diacetylenes) as shown by Pan and Charych (Langmuir 13: 1365 [1997]).

Ganglioside G_{M1}, cholera toxin from Vibrio Cholerae, human serum albumin, and wheat germ agglutinin can be purchased from Sigma. 5,7-docosadiynoic acid can be readily synthesized. Deionized water can be obtained by passing distilled water through a Millipore μF ultrapurification train. In preferred embodiments reagent grade solvents are used. The ganglioside G_{M1} is mixed at 5 mol % with the diacetylene “matrix lipid” monomers. Liposomes are prepared using the probe sonication method and polymerized by UV irradiation (254 nm). The conjugated ene-yne backbone of polydiacetylene liposomes result in the appearance of a deep blue/purple solution.

In one embodiment of the present invention, cholera toxin is diluted to 1 mg/ml in 50 mM Tris buffer, pH 7.0. In a 500 μl glass cuvette, blue phase liposomes produced as above are diluted 1:5 in 50 mM Tris buffer, pH 7.0. The liposomes are pre-incubated in the buffer for 15-30 minutes to ensure stability of the material prior to the addition of cholera toxin. Cholera toxin is added to the cuvette by the method of successive additions. After each addition, the contents are mixed and the fluorescence emission is recorded as a function of time. In preferred embodiments, the fluorescent response of the assay materials to addition of toxin (ligand) is practically instantaneous. After each experiment, the contents of the cuvette can be transferred to a single well of a microtiter plate to verify the binding of toxin by comparing the reacted assay materials to a negative control. It is contemplated that a negative response is observed if the ganglioside G_{M1} ligand is removed from the liposomes. Similarly, negative responses are obtained when comparable quantities of other proteins besides cholera toxin are added to the G_{M1}-containing liposomes.

In another embodiment of the present invention, toxin binding studies are carried out with the biosensor containing G_{M1} ganglioside in a buffer consisting of 50 mM Tris, 200 mM NaCl, 1 mM EDTA and 3 mM NaN₃, at pH 7.4. The films are first incubated in the buffer solution; films remaining in the buffer served as the background reference when toxins are added to generate a fluorometric response. The response time can be studied by monitoring the fluorescent emission of the biosensor immediately after the film is exposed to a solution containing 40 ppm cholera toxin and a three time points thereafter (e.g., 1 s (0.02 min), 30 min and 60 min.

III. Detection of E. coli Toxin

Liposomes are prepared with 5% by mole of G_{M1} and 95% 5,7-DCDA. E.coli toxin (Sigma) is spun through a 30 K molecular weight cutoff filter at 2000×g at 15° C. to remove salts. The protein is re-diluted in 50 mM Tris buffer pH 7.0 to a final concentration of 1 mg/ml. Liposomes are diluted in 50 mM Tris buffer, pH 8.0 to a final concentration of 0.2 mM in a plastic disposable cuvette. 40 μl of the above E. coli toxin is added to the liposome solution in the cuvette and fluorescent response is detected.

IV. Detection of Other Pathogens

The present invention may also be used to detect a variety of other pathogens. Ligands, specific for a large number of pathogens (e.g., carbohydrates, proteins, and antibodies) can be incorporated into the biopolymeric material using routine chemical synthesis methods described above and known in the art. Some of the examples of pathogen detection systems are presented below to demonstrate the variety of methods that can be applied using the present invention and to demonstrate the broad detecting capabilities of single ligand species (e.g., sialic acid).

The sialic acid derivated material of the present invention can be used to detect the presence of parasites such as Plasmodium (i.e., the etiologic agent that causes malaria). In these embodiments, the genetically conserved host binding site is utilized. PDA films containing sialic acid as described above are exposed to solutions containing malaria parasites and erythrocytes. After overnight exposure to the parasites, the fluorometric response of the assay materials are detected.

It is contemplated that the system can be used in conjunction with other testing material (e.g., arrays of biopolymeric material with various ligands) to identify and differentiate the presence of particularly virulent species or strains of Plasmodium (e.g., P. falciparum) or other pathogens.

In yet other embodiments, antibodies are used as ligands to successfully detect the presence of Neisseria gonorrhoeae and Vibrio vulnificus. The incorporation of the antibodies into the biopolymeric material is described in Example 5.

As is clear from these examples, the present invention provides a variety of means to detect a broad range of pathogens, including bacteria, viruses, and parasites.

V. Detection of Volatile Organic Chemicals (VOCs)

Certain embodiments of the presently claimed invention provide means to fluorescently detect volatile organic compounds (VOCs).

Two main approaches toward VOC detection have been adopted by various groups. The first involves traditional analytical techniques such as GC/MS that have been modified for VOC detection (i.e., an instrument-based approach) (Karpe et al., J. Chromatography A 708: 105 [1995]). However, these methods are expensive, complicated, and do not lend themselves to field or home use. The second involves the coupling of lipid membranes to detector surface(s) (i.e., an organic-device approach). In the past decade, several sensor devices that involve the coating of a piezoelectric mass balance with an organic film have been investigated. Because of the non-selective nature of the coating, these have been investigated in an array. These sensors, such as the quartz crystal microbalance (QCM) and the surface acoustic wave (SAW) devices (See e.g., Rose-Pehrsson et al., Anal. Chem. 60: 2801 [1988]), have linear frequency changes with applied mass. By applying a polymer or other coating to the crystal, a sensor based on the QCM or SAW is constructed. The complex electronics involved in the use of SAW, QCM, and electrode based systems makes these approaches less amenable to use as personal safety devices. The present invention differs from these methods in that signal transduction is an integral part of the organic layer structure rather than signal transduction to an electronic device.

The pharmaceutical industry has an ongoing need for solvent sensors, as pharmaceutical compounds are typically manufactured through organic chemical reactions that take place in the presence of solvents. Before packaging of a drug for use in humans or other animals, the solvent must be completely driven off (Carey and Kowalski, Anal. Chem. 60: 541 [1988]). The currently used method for detecting these VOCs uses energy intensive dryers to blow hot air across the drug and piezoelectric crystal arrays to analyze the evaporation of the various solvents (Carey, Trends in Anal. Chem. 13: 210 [1993]). The presently claimed invention provides a fluorometric based approach that greatly simplify these measurements.

VI. Other Small Organic Molecules

Certain inclusion compounds, or clathrates, have been shown to be highly selective sorbents for organic solvent vapors (See e.g., Ehlen, et al., Angew, Chem. Int. Ed. Engl. Vol. 32, p. 110 [1993]). The first step involves the synthesis of lipid diacetylene analogs of various clathrate compounds. The monomer-lipid clathrate is ordered and compressed on the water surface using a Langmuir-Blodgett film apparatus. Polymerization of the monolayer by UV irradiation yields is conducted as described above. The film is lifted onto a hydrophobized microscope slide. Exposure of these materials to analytes (e.g., 1-butanol or dioxane) produces a fluorometric response.

VII. Detection of Glucose with Hexokinase Ligands

The present invention contemplates the detection of glucose with hexokinase modified biopolymeric materials. Hexokinase modified biopolymeric materials are placed onto silanized glass cover slides. The biosensor coated glass cover slides are placed in glass cuvettes and the fluorescence of hexokinase modified films are recorded in 0.1 M phosphate buffer (pH 6.5). Measurements taken in this buffer condition are considered background. Glucose, or other sugar substitutes, are added directly ton the cuvettes. It is contemplated that the addition of glucose will provoke an immediate fluorometric response. The selectivity of the glucose sensor can be studied using sugar compounds structurally similar to glucose.

### VIII. Other Examples

The examples provided above demonstrate the broad range of analytes detectable by the presently claimed invention, ranging from complex biological organisms (e.g., viruses, bacteria, and parasites) to simple, small organic molecules (e.g., alcohols). The present invention contemplates that a number of other analytes can be successfully detected using ligands linked to biopolymeric material including, but not limited to botulinum neurotoxin detected with ganglioside incorporated PDA (Pan and Charych, Langmuir 13: 1367 [1997]). It is contemplated that numerous ligand types will be linked to monomers using standard chemical synthesis techniques known in the art to detect a broad range of analytes. Additionally, numerous other ligand types can be incorporated into the biopolymeric matrix without covalent attachment to monomer. These materials allow for the detection of small molecules, pathogens, bacteria, membrane receptors, membrane fragments, volatile organic compounds, enzymes, drugs, and many other relevant materials.

The presently claimed invention also finds use as a sensor in a variety of other applications. The fluorometric response of biopolymeric assay materials are affected by changes in temperature and pH. Thus, the methods and compositions of the presently claimed invention find use as temperature and pH detectors.

Ligands can also be used in the present invention when they function as competitive binders to the analyte. For example, by measuring the fluorometric/colorimetric response to an analyte in the presence of a natural receptor for the analyte, one can determine the quantity and/or binding affinity of the natural receptor. Application of competition or inhibition techniques allow the testing of very small, largely unreactive compounds, as well as substances present in very low concentrations or substances that have a small number or single valiancy. One application of this technique finds use as a means for the development and improvement of drugs by providing a screening assay to observe competitive inhibition of natural binding events. The present invention contemplates that the present compositions further provide a means for testing libraries of materials, as the binding of desired analytes is fluorometrically observed and the relevant biopolymeric material and its ligand can be separated from the others by segregating out a particular polymeric structure.

### EXAMPLE 8

### Immobilization of Biopolymeric Material

I. Immobilization to Silicon Chips and Gels

In one embodiment, silicon gel or wafers are acid cleaned in 1:1 HCl/methanol, rinsed in water, and placed in concentrated sulfuric acid. After a thorough water rinse, the wafer chips or gel is boiled in doubly distilled deionized water, allowed to cool and dry and then silanized under inert atmosphere in a 2% solution of 3-mercaptopropyl trimethoxysilane prepared in dry toluene. Next, the chips or gels are placed in a 2 mM solution of either GMBS (N-succinimidyl 4-maleimidobutyrate) or EMCS (N-succinimidyl 6-maleimidocaproate) prepared in 0.1 M phosphate buffer (the cross linker is first dissolved in a minimal amount of dimethylformamide). After rinsing with phosphate buffer, the chips are placed in a 0.05 mg/ml solution of the liposomes prepared in pH 8.0 phosphate buffer. Finally, the chips or gels are thoroughly rinsed with, and then stored in, the buffer solution prior to their use. The liposomes should have an —NH₂ functionality for the cross-linking with GMBS or EMCS to work.

II. Sol-Gel Entrapment of Biopolymeric Material

A silica sol is prepared by sonicating 15.25 g of tetramethylorthosilicate (TMOS), 3.35 g of water, and 0.22 ml of 0.04 N aqueous hydrochloric acid in a chilled bath until the solution is one phase (approximately 20 minutes). Chilled MOPS buffer solution (50% v/v) is then added to the acidic sol making sure that the solution is well cooled in an ice bath to retard gelation. A variety of materials are appropriate for generating silica sols, including, but not limited to, any tetraalkoxysilane or organically modified silane (e.g., ormosil). Additionally, tetraethylorthosilicate (TEOS), methyltriethoxysilane (MeTEOS), aryl silsesquioxanes, and other metal oxides find use in generating sol-gel glass.

For encapsulating liposomes, a polymerized liposome solution (2.5 ml) (as generated in Example 1) is then mixed into the buffered sol (10 ml) and the mixture poured into plastic cuvettes, applied as a film on a flat surface, or poured into any other desired formation template, sealed with Parafilm, and allowed to gel at ambient temperature. Gelation of the samples occurs within a few minutes resulting in transparent, monolithic solids (18 mm×10 mm×5 mm) in the case of cuvette formed gels and as violet colored monoliths with p-PDA liposomes. Slight shrinkage of aged monoliths could be observed due to syneresis.

The encapsulation of other biopolymeric material shapes (i.e., film and other nanostructures) can be conducted as described above. The materials is generated or sectioned into small (i.e., nanoscopic) sized portions if not already so, and incorporated into a solution to be mixed with the buffered sol.

### EXAMPLE 9

### Generation of Arrays

In some embodiments, the presently claimed invention contemplates the generation of a large palette of polymerizable lipids of different headgroup chemistries to create an array. Lipids containing head groups with carboxylic acid functionalities (imparting a formal negative charge), hydrophilic uncharged hydroxy groups, primary amine functionalities (that may acquire a formal positive charge), amino derivatives (with positive, negative or zwitterionic charge), and hydrophobic groups among others can be generated. In some embodiments of the present invention, the combination of these materials into a single device facilitates the simultaneous detection of a variety of analytes or the discrimination of a desired analytes from background interferants.

A large palette of polymerizable lipids of different headgroup chemistries can be generated to create an array. Suitable head group functionalities include, but are not limited to: 1) carboxylic acid functionalities, imparting a formal negative charge; 2) a hydrophilic uncharged hydroxyl group; 3) hydrophobic head groups; and 4) a primary amine that may acquire a formal positive charge. The amino acid derivatized assay materials may exist with positive, negative or zwitterionic charge.

Materials prepared as above, can be deposited into chambers of a device or immobilized to specific portions of a device. By generating biopolymeric materials with different properties (e.g., analyte detection capabilities, colors, analytes affinities, fluorescence emission properties) within a single apparatus (e.g., a badge), an array is generated with the ability to identify, distinguish, and quantitate analytes.

### EXAMPLE 10

### Modified Western Blot

In some embodiments, the biopolymeric materials of the present invention contain surface antibodies (See e.g., Example 5) that can be used in a Western blot procedure to detect the presence of proteins.

The protein samples (e.g., alpha-factor receptor) to be analyzed are solubilized with detergents and reducing agents, separated by SDS-polyacrylamide gel electrophoresis, and transferred to a solid support (e.g., nitrocellulose filter) using standard methods (See e.g., Sambrook et al, Molecular Cloning, A Laboratory Manual, 2nd ed., Cold Spring Harbor Laboratory Press, Plainview, N.Y., pp 18.60-18.75 [1989]). The nitrocellulose filter is placed in a heat-sealable plastic bag and 0.1 ml of blocking solution (e.g., 5% nonfat dried milk, 0.01% antifoam A, 0.02% sodium azide in phosphate-buffered saline) per square centimeter of filter is added. The bag is sealed, leaving as few air bubbles as possible, and incubated for 1-2 hours at room temperature with gentle agitation on a platform shaker.

The blocking solution is removed from the bag and a solution containing antibody-linked liposomes (i.e., prepared as described in Examples 1 and 5) is immediately added. The bag is resealed and incubated with gentle agitation for several minutes (although longer incubation times will typically increase the signal). The filter is removed from the bag and washed multiple times with phosphate buffered saline solution. No further steps are required, as the liposomes adhered to the protein on the membrane, providing a strong fluorescence signal. The advantage of this Western blot technique is the ease and speed with which protein can be detected. Unlike traditional Western techniques, there is no need for a secondary immunological reagent.

### Example 11

### Additional Fluorescence Microscopy Techniques

Studies of Polydiacetylene Films

General Instrumentation and Processing:

To examine the fluorescence properties of polydiacetylene films and vesicle suspensions with submicron resolution, an Olympus BH-2 microscope (Olympus America Inc., Melville, N.Y.) equipped with a 12-bit slow scan cooled Xillix CCD (Xillix Technologies Corp., Richmond, B.C., Canada) camera was used. Epi-fluorescence imaging was done using an Olympus rhodamine filter set (peak excitation 547 nm, emission filter 580 nm long pass) with a 100 watt mercury lamp illuminator. The 547 nm excitation filter gave the highest fluorescence output. The emission spectra gives two peaks at 557 and 618 nm with a tail that still gives 15% of the 557 maximun even at 700 nm.

The Xillix camera uses a Kodak KAF-1400 CCD chip with 1317×1035 pixels. Using 2×2 binning gives 650×516 pixel images with effective resolutions ranging from 1.3 μm to 0.22 μm depending on the objective. The 12 bit dynamic range allows for up to 4095 counts per pixel with shot noise error of less than 2% per image. Repeated images of the same fields can be summed to reduce noise even further. This is practical with PDL materials because of the very stable fluorescence of the red form that shows little bleaching even after several minutes of maximum intensity exposure with the mercury lamp. Image acquisition times for even weakly fluorescent materials was generally less than 1 sec., and as short as 15 msec. for fully red transformed polymers.

Langmuir Blodgget Films:

TRCDA Films. The fluorescence uniformity of tripentacosyl dienoic acid (TRCDA) (e.g., 10,12 diacetylene compositions) films was evaluated using a 20× 0.75 N.A. objective with a nominal resolution of 0.68 μm. Films were approximately 5×15 mm in size. Imaging of the films clearly revealed uniform domains extending hundreds of microns.

Sialic Acid Containing Films. TRCDA films made with the addition of 30% sialic acid residues produced initial count rates averaging 360±100 cps per pixel (˜0.5 um×0.5 um). After treating with 10 μl of a E-332:B/Panama flu virus suspension containing ˜8×10⁸ virus particles, the fluorescence intensities jumped more than 10 fold to an average of 4.2×10³ cps per pixel. This coincided with a visual change of the film from blue to pink corresponding to binding of the virus particles to the film via the sialic acid groups. In addition to the very large increase in fluorescence, the virus treated film appeared to take on a granular appearance. The number of viruses applied in these experiments, if fully bound, would correspond to a virus particle spacing of about 0.3 μm or about 10% coverage of the film. Since each virus contains about 1000 hemagglutinin trimers the number of bound sialyl groups per particle is expected to be very large. This factor taken together with the applied concentration of virus particles makes it plausible that significant mechanical stress results to the film. While not limited to any particular mechanism, the present invention contemplates in some embodiments, that the TRCDA film is buckled at submicron dimensions by the mechanical stress (shear forces) forces to produce a blue to red transition, this mode of action may in part or in whole generate the color and fluorescent changes when the films are treated with virus.

Vesicles Fluorescence Studies:

Total Internal Reflection Microscopy of Individual Flu-Vesicle Complexes. Experiments were conducted using total internal reflection microscopy (TIR) to visualize individual E332-flu viruses bound to sialyl-TRCDA. On the basis of spectrofluorometry, the quantum yield (QY) of the fluorescent red form of the polymer is comparatively weak compared to classic fluorophores. Initial estimates indicate that QYs for TRCDA polymers are more than two orders of magnitude smaller than for fluorescein on a per molecule basis. However, a single 50 nm vesicle will contain about 4.0×104 lipid monomers. Therefore, a single red transformed vesicle should provide a substantial signal especially when detected using ultrasensitive optical methods such as TIR, which can detect single molecules of rhodamine or fluorescein.

In some of these embodiments, the present invention contemplates visualizing individual (e.g., a single) red transformed vesicles. For example, with the mixing of vesicles to virus particles in a 1000 to 1 ratio, individual fluorescent objects were detected at the evanescent wave boundary excited in the TIR configuration, which nominally extends 100 to 200 nm into the vesicle suspension from the coverslip surface. The optical assembly included a 100×1.4 N.A. objective, a 532 nm 100 mW laser for illumination, and a NeuroCam 80×80 (A.M.I. Tech., Hod Hasharon, Israel) pixel back illuminated CCD for fluorescence detection. The NeuroCam CCD uses 24 μm pixels that are ˜75% quantum efficient in the emission range of the polymer. Using the 100× objective gives a sampling size of 0.24 μm per pixel so that particles in the suboptical size range (<0.24 μm) will produce spots with FWHM sizes of approximately three pixels. This is due to spatial undersampling by the CCD.

Using 6 msec. exposure times, peak pixel counts for individual fluorescent particles ranged from 1000 to 4000 counts per pixel per 6 msec. with FWHM diameters of three pixels (0.72 μm), which is basically the resolution limit of the system. To estimate the number of vesicles that corresponds to a given fluorescent spot, it was assumed that a single vesicle contains ˜4×104 lipids with a QY equivalent to about 40 rhodamine molecules. The latter was calibrated on the TIR and shown to give ˜3×10⁴ counts in a 6 msec. exposure for a 3×3 pixel area. Integration of the flu-vesicle spot intensities gave values ranging from 2.0-6.0×10⁴ indicating that the spots correspond to about one to three fully transformed vesicles. Considering the spot sizes detected, the fluorescent domain of the fluorescent particle cannot be larger than 0.24 μm, this corresponds to a viral particle surrounded by a single layer of fluorescent vesicles.

Instrument Development for Ultrasensitive Detection:

Film Screening. The present invention demonstrates using conventional CCD wide field camera instrumentation to quantify fluorescence intensity variations over broad areas of polymerized planar films revealing crystalline domains hundreds of microns in size, as well as, local variations at the submicron level. For the production of consistent high quality films, screening of large film areas (0.5×1.5 cm) is required. To accomplish this, an automated image acquisition system using a computer controlled scanning stage is used. Using a 20× 0.75 NA lens (0.6 μm resolution), approximately 75 images would be required to analyze a 0.5×1.5 cm film and would take about 75 Mbytes per specimen of disk storage. With the scanning stage the imaging can be done automatically with near real time image analysis using a fast work station.

Polymer Physical Chemistry and Structural Analysis. While not limited to any particular mechanism, the present invention contemplates that the intense fluorescence changes observed when PDA is transformed, provides crucial information for elucidating the mechanism of the film transformation. Single molecules detection system using total internal reflection microscopy (TIR) is used to measure the conversion of single 50 nm size vesicles by flu virus into fluorescent reporters. The present invention quantifies the transformation of single vesicles with errors of only a few percent permitting an accurate characterization of the vesicle-flu particle interaction. In addition, to the TIR systems used herein, other embodiments use confocal or NSOM Piezo scanning stage system to make accurate fluorescent domain propagation analysis of flu bound to monolayer films. For example, it was determined that the fluorescence intensities of flu treated polymers at the 0.22 μm level can be measured with high accuracy using these systems.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in material science, chemistry, and molecular biology or related fields are intended to be within the scope of the following claims.

We claim:

1. A method for detecting a reaction, comprising:
a) providing:
i) biopolymeric material comprising reaction substrate and a plurality of self-assembling monomers; and
ii) a reaction means;
b) exposing said reaction means to said biopolymeric material; and
c) detecting an induced fluorescence emission in said biopolymeric material which indicates at least a partial occurrence of said reaction.

2. The method of claim 1, wherein said reaction means comprises a lipid cleavage means.

3. The method of claim 1, further comprising the step of quantifying said color change in said biopolymeric material.

4. The method of claim 1, wherein said biopolymeric materials are selected from the group consisting of liposomes, films, tubules, helical assemblies, fiber-like assemblies, and solvated polymers.

5. The method of claim 1, wherein said self assembling monomers comprise diacetylene monomers.

6. The method of claim 1, wherein said self assembling monomers comprise diacetylene monomers selected from the group consisting of 5,7-docosadiynoic acid, 5,7-pentacosadiynoic acid, 10,12-pentacosadiynoic acid, and combinations thereof.

7. The method of claim 1, wherein said self-assembling monomers are selected from the group consisting of acetylenes, alkenes, thiophenes, polythiophenes, siloxanes, polysilanes, anilines, pyrroles, polyacetylenes, poly (para-phylenevinylene), poly (para-phylene), vinylpyridinium, and combinations thereof.

8. The method of claim 1, wherein said biopolymeric material further comprises one or more ligands.

9. The method of claim 8, wherein said one or more ligands is selected from the group consisting of proteins, antibodies, carbohydrates, nucleic acids, drugs, chromophores, antigens, chelating compounds, short peptides, pepstatin, Diels-Alder reagents, molecular recognition complexes, ionic groups, polymerizable groups, linker groups, electron donors, electron acceptor groups, hydrophobic groups, hydrophilic groups, receptor binding groups, trisaccharides, tetrasaccharides, ganglioside G_{M1}, ganglioside G_{T1b}, sialic acid, and combinations thereof.

10. The method of claim 8, wherein said one or more ligands have affinity for said reaction means.

11. The method of claim 1, wherein said biopolymeric material further comprises one or more dopants.

12. The method of claim 11, wherein said one or more dopants is selected from the group consisting of surfactants, polysorbate, octoxynol, sodium dodecyl sulfate, polyethylene glycol, zwitterionic detergents, decylglucoside, deoxycholate, diacetylene derivatives, phosphatidylserine, phosphatidylinositol, phosphatidylethanolamine, phosphatidylcholine, phosphatidylglycerol, phosphatidic acid, phosphatidylmethanol, cardiolipin, ceramide, cholesterol, steroids, cerebroside, lysophosphatidylcholine, D-erythroshingosine, sphingomyelin, dodecyl phosphocholine, N-biotinyl phosphatidylethanolamine and combinations thereof.

13. The method of claim 11, wherein said one or more dopants comprises diacetylene derivatives selected from the group consisting of sialic acid-derived diacetylene, lactose-derived diacetylene, amino acid-derived diacetylene, and combinations thereof.

14. The method of claim 1, wherein said biopolymeric material further comprises a support, and wherein said biopolymeric material is immobilized to said support.

15. The method of claim 14, wherein said support is selected from the group consisting of polystyrene, polyethylene, teflon, mica, sephadex, sepharose, polyacrynitriles, filters, glass, gold, silicon chips, and silica.

16. The method of claim 2, wherein said cleavage means comprises a lipase.

17. The method of claim 16, wherein said lipase is selected from the group consisting of phospholipase A₂, phospholipase C, and phospholipase D.

18. A method for detecting the presence of an analyte, comprising providing biopolymeric material comprising analyte substrate and a plurality of self-assembling monomers; exposing a sample suspected of containing said analyte to said biopolymeric material; and detecting an induced fluorescence emission.

19. The method of claim 18, wherein said analyte comprises a lipid cleavage means.

20. The method of claim 18, wherein said biopolymeric materials are selected from the group consisting of liposomes, films, tubules, helical assemblies, fiber-like assemblies, and solvated polymers

21. The method of claim 18, wherein said self assembling monomers comprise diacetylene monomers.

22. The method of claim 18, wherein said self-assembling monomers comprise diacetylene monomers selected from the group consisting of 5,7-docosadiynoic acid, 5,7-pentacosadiynoic acid, 10,12-pentacosadiynoic acid, and combinations thereof.

23. The method of claim 18, wherein said self-assembling monomers are selected from the group consisting of acetylenes, alkenes, thiophenes, polythiophenes, siloxanes, polysilanes, anilines, pyrroles, polyacetylenes, poly (para-phylenevinylene), poly (para-phylene), vinylpyridinium, and combinations thereof.

24. The method of claim 18, wherein said biopolymeric material further comprises one or more ligands.

25. The method of claim 24, wherein said one or more ligands is selected from the group consisting of proteins, antibodies, carbohydrates, nucleic acids, drugs, chromophores, antigens, chelating compounds, short peptides, pepstatin, Diels-Alder reagents, molecular recognition complexes, ionic groups, polymerizable groups, linker groups, electron donors, electron acceptor groups, hydrophobic groups, hydrophilic groups, receptor binding groups, trisaccharides, tetrasaccharides, ganglioside G_{M1}, ganglioside G_{T1b}, sialic acid, and combinations thereof.

26. The method of claim 24, wherein said one or more ligands have affinity for said analyte.

27. The method of claim 18, wherein said biopolymeric material further comprises one or more dopants.

28. The method of claim 27, wherein said one or more dopants is selected from the group consisting of surfactants, polysorbate, octoxynol, sodium dodecyl sulfate, polyethylene glycol, zwitterionic detergents, decylglucoside, deoxycholate, diacetylene derivatives, phosphatidylserine, phosphatidylinositol, phosphatidylethanolamine, phosphatidylcholine, phosphatidylglycerol, phosphatidic acid, phosphatidylmethanol, cardiolipin, ceramide, cholesterol, steroids, cerebroside, lysophosphatidylcholine, D-erythroshingosine, sphingomyelin, dodecyl phosphocholine, N-biotinyl phosphatidylethanolamine and combinations thereof.

29. The method of claim 27, wherein said one or more dopants comprises diacetylene derivatives selected from the group consisting of sialic acid-derived diacetylene, lactose-derived diacetylene, amino acid-derived diacetylene, and combinations thereof.

30. The method of claim 18, wherein said biopolymeric material further comprises a support, and wherein said biopolymeric material is immobilized to said support.

31. The method of claim 30, wherein said support is selected from the group consisting of polystyrene, polyethylene, teflon, mica, sephadex, sepharose, polyacrynitriles, filters, glass, gold, silicon chips, and silica.

32. The method of claim 19, wherein said cleavage means comprises a lipase.

33. The method of claim 32, wherein said lipase is selected from the group consisting of phospholipase A₂, phospholipase C, and phospholipase D.

34. A method for detecting inhibitors, comprising:
a) providing:
i) biopolymeric material comprising reaction substrate and a plurality of self-assembling monomers;
ii) a reaction means; and
iii) a sample suspected of containing an inhibitor;
b) combining said biopolymeric material and said sample suspected of containing an inhibitor;
c) exposing said biopolymeric material and said sample suspected of containing an inhibitor to said reaction means; and
d) detecting induced fluorescence emission in said biopolymeric material, thereby detecting the activity of said inhibitor.

35. The method of claim 34, wherein said detecting a color change in said biopolymeric material comprises comparing said color change to one or more control samples.

36. The method of claim 34, further comprising the step of quantitating said color change in said biopolymeric material.

37. The method of claim 34, wherein said reaction means comprises a cleavage means.

38. The method of claim 34, wherein said biopolymeric materials are selected from the group consisting of liposomes, films, tubules, helical assemblies, fiber-like assemblies, and solvated polymers

39. The method of claim 34, wherein said self assembling monomers comprise diacetylene monomers.

40. The method of claim 34, wherein said self-assembling monomers comprise diacetylene monomers selected from the group consisting of 5,7-docosadiynoic acid, 5,7-pentacosadiynoic acid, 10,12-pentacosadiynoic acid, and combinations thereof.

41. The method of claim 34, wherein said self-assembling monomers are selected from the group consisting of acetylenes, alkenes, thiophenes, polythiophenes, siloxanes, polysilanes, anilines, pyrroles, polyacetylenes, poly (para-phylenevinylene), poly (para-phylene), vinylpyridinium, and combinations thereof.

42. The method of claim 34, wherein said biopolymeric material further comprises one or more ligands.

43. The method of claim 42, wherein said one or more ligands is selected from the group consisting of proteins, antibodies, carbohydrates, nucleic acids, drugs, chromophores, antigens, chelating compounds, short peptides, pepstatin, Diels-Alder reagents, molecular recognition complexes, ionic groups, polymerizable groups, linker groups, electron donors, electron acceptor groups, hydrophobic groups, hydrophilic groups, receptor binding groups, trisaccharides, tetrasaccharides, ganglioside G_{M1} ganglioside G_{T1b}, sialic acid, and combinations thereof.

44. The method of claim 42, wherein said one or more ligands have affinity for said reaction means.

45. The method of claim 34, wherein said biopolymeric material further comprises one or more dopants.

46. The method of claim 45, wherein said one or more dopants is selected from the group consisting of surfactants, polysorbate, octoxynol, sodium dodecyl sulfate, polyethylene glycol, zwitterionic detergents, decylglucoside, deoxycholate, diacetylene derivatives, phosphatidylserine, phosphatidylinositol, phosphatidylethanolamine, phosphatidylcholine, phosphatidylglycerol, phosphatidic acid, phosphatidylmethanol, cardiolipin, ceramide, cholesterol, steroids, cerebroside, lysophosphatidylcholine, D-erythroshingosine, sphingomyelin, dodecyl phosphocholine, N-biotinyl phosphatidylethanolamine and combinations thereof.

47. The method of claim 45, wherein said one or more dopants comprise diacetylene derivatives selected from the group consisting of sialic acid-derived diacetylene, lactose-derived diacetylene, amino acid-derived diacetylene, and combinations thereof.

48. The method of claim 34, wherein said biopolymeric material further comprises a support, and wherein said biopolymeric material is immobilized to said support.

49. The method of claim 48, wherein said support is selected from the group consisting of polystyrene, polyethylene, teflon, mica, sephadex, sepharose, polyacrynitriles, filters, glass, gold, silicon chips, and silica.

50. The method of claim 37, wherein said cleavage means comprises a lipase.

51. The method of claim 50, wherein said lipase is selected from the group consisting of phospholipase A₂, phospholipase C, and phospholipase D.

52. An array comprising a plurality of polymer molecules comprising biopolymeric material and one or more ligands wherein the binding of an analyte to said one or more ligands produces an induced fluorescence emission in said biopolymeric material.

53. The array of claim 52, wherein said analyte comprises a virus particle.
